(19) United States
(12) Patent Application Publication
(10) Pub. No.: US 2024/0294971 A1
(43) Pub. Date: Sep. 5, 2024

(54) METHODS FOR SPATIAL ANALYSIS USING DNA CAPTURE

(71) Applicant: 10x Genomics, Inc., Pleasanton, CA (US)

(72) Inventor: Christina Galonska, Stockholm (SE)

(21) Appl. No.: 18/662,431

(22) Filed: May 13, 2024

Related U.S. Application Data
(62) Division of application No. 17/406,824, filed on Aug. 19, 2021, now Pat. No. 11,981,958.
(60) Provisional application No. 63/068,146, filed on Aug. 20, 2020.

(51) Int. Cl. C12Q 1/6827; C12Q 1/6876

(52) U.S. Cl. CPC C12Q 1/6827; C12Q 1/6876; C12Q 2600/156

(57) ABSTRACT

Provided herein are methods of detecting an analyte of interest to interrogate spatial gene expression in a sample using DNA templated ligation. For example, provided herein are methods for detecting an gDNA analyte in a biological sample where (i) the first RTL probe and second RTL probe hybridize to adjacent sequences on the gDNA analyte, (ii) enzyme-mediate cleavage of a 5′ FLAP results in release of the 5′ FLAP, and (iii) the sequence of the 5′ FLAP is determined and used to detect the gDNA analyte in the biological sample.

### CROSS-REFERENCE TO RELATED APPLICATIONS

This application is a divisional application of patent application Ser. No. 17/406,824, filed Aug. 19, 2021, which claims priority to U.S. Provisional Patent Application No. 63/068,146, filed Aug. 20, 2020. The entire content of the foregoing application is incorporated herein by reference.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Spatial analysis takes advantage of targeting a particular analyte in a sample using a capture probe that includes a capture domain capable of capturing the particular analyte. In the context of RNA, one approach developed to enhance resolution of spatial analysis is a process called RNA-templated ligation (RTL), which includes multiple oligonucleotides targeting adjacent or nearby complementary sequences on the analyte (e.g., RNA). RTL methods have not been adapted to different types of nucleic acid analytes in the context of spatial analysis. Thus, there remains a need to adapt RTL methods to the spatial analysis of a genomic DNA (gDNA) molecule.

### SUMMARY

Featured herein are methods of identifying a target gDNA analyte of interest or a genetic variant in a gDNA analyte. In particular, methods provided herein co-opt traditional RTL principles to include oligonucleotide probes that hybridize to adjacent complementary genomic DNA (gDNA) sequences. Methods provided herein allow a readout of a DNA-templated hybridization event that does not require single stranded DNA from the DNA-DNA template. For example, an oligonucleotide (e.g., a second RTL probe) that hybridizes to the gDNA analyte includes a 5′ FLAP sequence that can be removed and detected upon an enzyme-mediated cleavage event that does not require removal of the hybridized oligonucleotide probes. Detection of the 5′ FLAP provides a readout for the DNA-templated hybridization event, which can serve as a proxy for detecting a gDNA analyte or a genetic variant within a gDNA analyte.

In one aspect, the disclosure features a method for determining the abundance and/or location of a genomic DNA (gDNA) analyte in a biological sample, the method comprising: (a) contacting the biological sample with a substrate comprising a plurality of capture probes, wherein a capture probe of the plurality comprises a spatial barcode and a capture domain; (b) contacting the biological sample with a first RTL probe and a second RTL probe, wherein the first RTL probe and the second RTL probe are substantially complementary to adjacent sequences of the gDNA analyte, and wherein the second RTL probe comprises a 5′ FLAP; (c) hybridizing the first RTL probe and the second RTL probe to the gDNA analyte; (d) cleaving the second RTL probe, thereby releasing the 5′ FLAP; (e) hybridizing the 5′ FLAP to the capture domain; and (f) determining (i) all or a part of the sequence of the 5′ FLAP, or a complement thereof, and (ii) all or a part of the sequence of the spatial barcode, or a complement thereof, and using the determined sequence of (i) and (ii) to identify the abundance and/or location of the gDNA analyte in the biological sample.

In some embodiments, the first RTL probe and the second RTL probes are DNA probes.

In some embodiments, the 5′ FLAP comprises a first barcode sequence, wherein the first barcode sequence comprises a sequence that identifies the first RTL probe, the second RTL probe, the gDNA analyte, or any combination thereof.

In some embodiments, the 5′ FLAP comprises (i) a functional sequence, wherein the functional sequence is a primer sequence, and (ii) a capture probe binding domain, wherein the capture probe binding domain comprises a homopolymeric sequence or a poly(A) sequence.

In some embodiments, the cleaving the second RTL probe comprises providing an endonuclease, wherein the endonuclease cleaves a portion of the first RTL probe, a portion of second RTL probe, the 5′ FLAP of the second RTL probe, or any combination thereof.

In some embodiments, the determining step comprises sequencing all or part of the 5′ FLAP.

In some embodiments, the biological sample is a formalin-fixed, paraffin-embedded (FFPE) sample.

In another aspect, the disclosure features a method of detecting a genetic variant in a gDNA analyte in a biological sample, the method comprising: (a) contacting the biological sample with a substrate comprising a plurality of capture probes, wherein a capture probe of the plurality comprises a spatial barcode and a capture domain; (b) contacting the biological sample with a first RTL probe and a second RTL probe, wherein the first RTL probe and the second RTL probe each comprise a sequence that is substantially complementary to adjacent sequences of the gDNA analyte; wherein the first RTL probe and the second RTL probe are capable of forming an invasive cleavage structure in the presence of the genetic variant; and wherein the second RTL probe further comprises a 5′ FLAP; (c) hybridizing the first RTL probe and the second RTL probe to the gDNA analyte; (d) cleaving the second RTL probe when the genetic variant is present, thereby releasing the 5′ FLAP; (e) hybridizing the 5′ FLAP to the capture domain; and (f) determining (i) all or a part of the sequence of the 5′ FLAP, or a complement thereof, and (ii) all or a part of the sequence of the spatial barcode, or a complement thereof, and using the determined sequence of (i) and (ii) to detect the genetic variant in the gDNA analyte in the biological sample.

In some embodiments, the genetic variant is a single nucleotide polymorphism (SNP) or a nucleotide point mutation, or wherein the genetic variant comprises at least two, at least three, at least four, at least five, or more genetic variants.

In some embodiments, the first RTL probe comprises a sequence that is substantially complementary to a sequence 3′ to the genetic variant, or at least one nucleotide that is complementary to a wild-type sequence of the genetic variant.

In some embodiments, the second RTL probe comprises a sequence substantially complementary to a sequence 5′ to the genetic variant, and/or a nucleotide that is complementary to the genetic variant.

In some embodiments, the 5′ FLAP comprises a nucleotide that is complementary to the genetic variant.

In some embodiments, the first RTL probe and the second RTL probe are capable of forming an invasive cleavage structure in the presence of the genetic variant.

In some embodiments, the 5′ FLAP comprises a capture probe binding domain, wherein the capture probe binding domain comprises a homopolymeric sequence or a poly(A) sequence.

In some embodiments, the 5′ FLAP comprises from 5′ to 3′; a functional sequence, a barcode, and a capture probe binding domain sequence.

In some embodiments, the 5′ FLAP comprises from 5′ to 3′; a functional sequence, a barcode, a capture probe binding domain sequence, and an additional nucleotide, wherein the additional nucleotide comprises a nucleotide that is complementary to the genetic variant or a nucleotide that is complementary to the wild type sequence of the genetic variant.

In some embodiments, the method further comprises providing a capture probe binding domain blocking moiety that interacts with the capture probe binding domain, wherein the method further comprises releasing the capture probe binding domain blocking moiety from the capture probe binding domain prior to contacting the biological sample with the substrate, wherein the capture probe binding domain blocking moiety comprises a poly-uridine sequence, a poly-thymidine sequence, or both, and wherein releasing the capture probe binding domain blocking moiety from the poly(A) sequence comprises denaturing the ligated probe.

In some embodiments, the biological sample is an FFPE sample.

In another aspect, the disclosure features a kit comprising: (a) a first RTL probe and a second RTL probe, wherein the first RTL probe and the second RTL probe are substantially complementary to adjacent sequences of a gDNA analyte, or wherein the first RTL probe and the second RTL probe each comprise a sequence that is substantially complementary to adjacent sequences of the gDNA analyte, and wherein the second RTL probe comprises a 5′ FLAP; (b) an endonuclease, wherein the endonuclease cleaves the 5′ FLAP thereby releasing the 5′ FLAP from the second RTL probe; (c) a substrate comprising a plurality of capture probes, wherein a capture probe of the plurality comprises a spatial barcode and a capture domain; and (d) instructions for performing any one of the methods described herein.

In another aspect, the disclosure features a composition for determining the abundance and/or location of a gDNA analyte in a biological sample, wherein the composition comprises: a first RTL probe and a second RTL probe hybridized to the gDNA analyte, wherein the first RTL probe and the second RTL probe are substantially complementary to adjacent sequences of the gDNA analyte, or wherein the first RTL probe and the second RTL probe each comprise a sequence that is substantially complementary to adjacent sequences of the gDNA analyte, and wherein the second RTL probe comprises a 5′ FLAP, wherein the 5′ FLAP comprises a sequence that is capable of hybridizing to a capture domain of a capture probe.

In one aspect, this disclosure features methods for detecting a genomic DNA (gDNA) molecule in a biological sample, including: (a) contacting the biological sample with a first probe oligonucleotide and a second probe oligonucleotide, wherein the first probe oligonucleotide and the second probe oligonucleotide are substantially complementary to adjacent sequences of the gDNA molecule, and wherein the second probe oligonucleotide includes a 5′ FLAP; (b) hybridizing the first probe oligonucleotide and the second probe oligonucleotide to the gDNA molecule; (c) cleaving the second probe oligonucleotide, thereby releasing the 5′ FLAP; and (d) determining all or a part of the sequence of the 5′ FLAP to detect the gDNA molecule.

In another aspect, this disclosure features methods of detecting a genetic variant in a gDNA molecule in a biological sample including: (a) contacting the biological sample with a first probe oligonucleotide and a second probe oligonucleotide, wherein the first probe oligonucleotide and the second probe oligonucleotide each include a sequence that is substantially complementary to adjacent sequences of the gDNA molecule; wherein the first probe oligonucleotide and the second probe oligonucleotide are capable of forming an invasive cleavage structure in the presence of the genetic variant; and wherein the second probe oligonucleotide further includes a 5′ FLAP; (b) hybridizing the first probe oligonucleotide and the second probe oligonucleotide to the gDNA molecule; (c) cleaving the second probe oligonucleotide when the genetic variant is present, thereby releasing the 5′ FLAP; and (d) determining all or a part of the sequence of the 5′ FLAP to detect the genetic variant in the gDNA molecule.

In some embodiments, the gDNA molecule includes one or more single nucleotide variants compared to a reference gDNA molecule.

In some embodiments, the first probe oligonucleotide includes a sequence that is substantially complementary to a sequence 3′ to the genetic variant. In some embodiments, the first probe oligonucleotide further includes at least one nucleotide that is complementary to a wild-type sequence of the genetic variant. In some embodiments, the first probe oligonucleotide includes at least two ribonucleic acid bases at the 3′ end. In some embodiments, the first probe oligonucleotide includes at least two deoxyribonucleic acid bases at the 3′ end.

In some embodiments, the second probe oligonucleotide includes a sequence substantially complementary to a sequence 5′ to the genetic variant.

In some embodiments, the 5′ FLAP further includes a first barcode sequence. In some embodiments, the first barcode sequence includes a sequence that identifies at least one of the first probe oligonucleotide, the second probe oligonucleotide, or the gDNA molecule.

In some embodiments, the 5′ FLAP further includes a functional sequence. In some embodiments, the functional sequence is selected from a primer sequence, a Read 1 sequence, a Read 2 sequence, an index sequence, a P5 index sequence, and a P7 index sequence.

In some embodiments, the 5′ FLAP further includes a nucleotide that is complementary to the genetic variant. In some embodiments, the second probe oligonucleotide further includes a second barcode.

In some embodiments, the second probe oligonucleotide includes a sequence that is complementary to a capture domain. In some embodiments, the second probe oligonucleotide further includes a nucleotide that is complementary to the genetic variant.

In some embodiments, the cleaving step includes providing an endonuclease. In some embodiments, the endonuclease cleaves the invasive cleavage structure.

In some embodiments, the endonuclease cleaves a portion of the first probe oligonucleotide. In some embodiments, the endonuclease cleaves the at least one nucleotide that is complementary to a wild-type sequence of the gDNA molecule of the first probe oligonucleotide. In some embodiments, the endonuclease cleaves a portion of second probe oligonucleotide. In some embodiments, the endonuclease cleaves the 5′ FLAP of the second probe oligonucleotide. In some embodiments, the endonuclease is FLAP endonuclease 1 (FEN1).

In some embodiments, the method further includes contacting the biological sample with a permeabilization reagent.

In some embodiments, the biological sample is contacted with the permeabilization reagent before step (d) (e.g., determining all or a part of the sequence of the 5′ FLAP to detect the gDNA molecule).

In some embodiments, the method further includes denaturing the gDNA under conditions wherein the first probe oligonucleotide and the second probe oligonucleotide can hybridize to the gDNA molecule.

In some embodiments, the determining step includes amplifying all or part of the 5′ FLAP. In some embodiments, the amplifying is isothermal. In some embodiments, the amplifying is not isothermal. In some embodiments, the determining step includes sequencing.

In another aspect, this disclosure features methods for detecting a gDNA molecule at a spatial location in a biological sample, including: (a) contacting a biological sample with a first probe oligonucleotide and a second probe oligonucleotide, wherein the first probe oligonucleotide and the second probe oligonucleotide are substantially complementary to adjacent sequences of the gDNA molecule, and wherein the second probe oligonucleotide includes a 5′ FLAP; (b) hybridizing the first probe oligonucleotide and the second probe oligonucleotide to the gDNA molecule; (c) cleaving the second probe oligonucleotide, thereby releasing the 5′ FLAP; (d) contacting the biological sample with a substrate including a plurality of capture probes, wherein a capture probe of the plurality includes a spatial barcode and a capture domain; (e) hybridizing the 5′ FLAP to the capture domain; and (f) determining (i) all or a part of the sequence of the 5′ FLAP, or a complement thereof, and (ii) all or a part of the sequence of the spatial barcode, or a complement thereof, and using the determined sequence of (i) and (ii) to identify the spatial location of the gDNA molecule in the biological sample.

In another aspect, this disclosure features methods of detecting a genetic variant in a genomic DNA (gDNA) molecule at a spatial location in a biological sample including: (a) contacting the biological sample with a first probe oligonucleotide and a second probe oligonucleotide, wherein the first probe oligonucleotide and the second probe oligonucleotide each include a sequence that is substantially complementary to adjacent sequences of the gDNA molecule; wherein the first probe oligonucleotide and the second probe oligonucleotide are capable of forming an invasive cleavage structure in the presence of the genetic variant; and wherein the second probe oligonucleotide further includes a 5′ FLAP; (b) hybridizing the first probe oligonucleotide and the second probe oligonucleotide to the gDNA molecule; (c) cleaving the second probe oligonucleotide when the genetic variant is present, thereby releasing the 5′ FLAP; (d) contacting the biological sample with a substrate including a plurality of capture probes, wherein a capture probe of the plurality includes a spatial barcode and a capture domain; (e) hybridizing the cleavage product to the capture domain; and (f) determining (i) all or a part of the sequence of the 5′ FLAP, or a complement thereof, and (ii) all or a part of the sequence of the spatial barcode, or a complement thereof, and using the determined sequence of (i) and (ii) to identify the spatial location of the gDNA molecule in the biological sample.

In some embodiments, the gDNA molecule includes one or more single nucleotide variants compared to a reference gDNA molecule.

In some embodiments, the first probe oligonucleotide includes a sequence that is substantially complementary to a sequence 3′ of the genetic variant. In some embodiments, the first probe oligonucleotide further includes at least one nucleotide that is complementary to a wild-type sequence of the genetic variant. In some embodiments, the first probe oligonucleotide includes at least two ribonucleic acid bases at the 3′ end. In some embodiments, the first probe oligonucleotide includes at least two deoxyribonucleic acid bases at the 3′ end.

In some embodiments, the second probe oligonucleotide includes a sequence substantially complementary to a sequence 5′ to the genetic variant.

In some embodiments, the 5′ FLAP further includes a first barcode sequence. In some embodiments, the first barcode sequence includes a sequence that identifies at least one of the first probe oligonucleotide, the second probe oligonucleotide, or the gDNA molecule. In some embodiments, the first barcode sequence includes a sequence that identifies the gDNA molecule.

In some embodiments, the 5′ FLAP further includes a functional sequence. In some embodiments, the functional sequence is selected from a primer sequence, a Read 1 sequence, a Read 2 sequence, an index sequence, a P5 index sequence, and a P7 index sequence.

In some embodiments, the 5′ FLAP further includes a capture probe binding domain. In some embodiments, the capture probe binding domain includes a homopolymeric sequence. In some embodiments, the capture probe binding domain includes a poly(A) sequence. In some embodiments, the 5′ FLAP further includes a nucleotide that is complementary to the genetic variant.

In some embodiments, the 5′ FLAP includes from 5′ to 3′; a functional sequence, a barcode, and a capture probe binding domain sequence. In some embodiments, the 5′ FLAP includes from 5′ to 3′; a functional sequence, a barcode, a capture probe binding domain sequence, and an additional nucleotide. In some embodiments, the additional nucleotide includes a nucleotide that is complementary to the genetic variant. In some embodiments, the additional nucleotide includes a nucleotide that is complementary to the wild type sequence of the genetic variant.

In some embodiments, the method further includes providing a capture probe binding domain blocking moiety that interacts with the capture probe binding domain.

In some embodiments, the method further includes releasing the capture probe binding domain blocking moiety from the capture probe binding domain prior to contacting the biological sample with the substrate. In some embodiments, the capture probe binding domain blocking moiety includes a poly-uridine sequence, a poly-thymidine sequence, or both. In some embodiments, releasing the capture probe binding domain blocking moiety from the poly(A) sequence includes denaturing the ligated probe.

In some embodiments, the second probe oligonucleotide further includes a second barcode.

In some embodiments, the second probe oligonucleotide further includes a second capture probe binding domain.

In some embodiments, the second probe oligonucleotide further includes a nucleotide that is complementary to the genetic variant.

In some embodiments, the cleaving step includes providing an endonuclease. In some embodiments, the endonuclease cleaves the invasive cleavage structure. In some embodiments, the endonuclease cleaves a portion of the first probe oligonucleotide. In some embodiments, the endonuclease cleaves the at least one nucleotide that is complementary to a wild-type sequence of the gDNA molecule of the first probe oligonucleotide. In some embodiments, the endonuclease cleaves a portion of second probe oligonucleotide. In some embodiments, the endonuclease cleaves the 5′ FLAP of the second probe oligonucleotide. In some embodiments, the endonuclease is FLAP endonuclease 1 (FEN1).

In some embodiments, the method further includes contacting the biological sample with a permeabilization reagent. In some embodiments, the biological sample is contacted with the permeabilization reagent before step (d) (e.g., contacting the biological sample with a substrate including a plurality of capture).

In some embodiments, the method further includes denaturing the gDNA under conditions wherein the first probe oligonucleotide and the second probe oligonucleotide can hybridize to the gDNA molecule.

In some embodiments, the determining step includes amplifying all or part of the 5′ FLAP specifically bound to the capture domain. In some embodiments, the amplifying is isothermal. In some embodiments, the amplifying is not isothermal. In some embodiments, the determining step includes sequencing.

In some embodiments of any of the methods for detecting a genetic variant in a gDNA molecule, the genetic variant is a single nucleotide polymorphism (SNP). In some embodiments of any of the methods for detecting a genetic variant in a gDNA molecule, the genetic variant is a nucleotide point mutation. In some embodiments of any of the methods for detecting a genetic variant in a gDNA molecule, the genetic variant includes at least two, at least three, at least four, at least five, or more genetic variants.

In some embodiments, the first probe oligonucleotide is at least 70%, at least 75%, at least 80%, at least 85%, at least 90%, at least 95%, at least 96%, at least 97%, at least 98%, at least 99%, or 100% complementary to a sequence of the gDNA molecule. In some embodiments, the second probe oligonucleotide is at least 70%, at least 75%, at least 80%, at least 85%, at least 90%, at least 95%, at least 96%, at least 97%, at least 98%, at least 99%, or 100% complementary to a sequence of the gDNA molecule.

In some embodiments, the biological sample is a formalin-fixed, paraffin-embedded (FFPE), frozen, or fresh sample. In some embodiments, the biological sample is a FFPE sample.

In another aspect, this disclosure features a kit for use in a method of detecting analyte in a biological sample, wherein the kit includes any two or more of: (a) a first probe oligonucleotide and a second probe oligonucleotide, wherein the first probe oligonucleotide and the second probe oligonucleotide are substantially complementary to adjacent sequences of the analyte, and wherein the second probe oligonucleotide includes a 5′ FLAP; (b) an endonuclease, wherein the endonuclease cleaves the 5′ FLAP thereby releasing the 5′ FLAP from the second probe oligonucleotide; (c) a substrate including a plurality of capture probes, wherein a capture probe of the plurality includes a spatial barcode and a capture domain, wherein the 5′ FLAP is capable of hybridizing to the capture domain; and (d) instructions for carrying out any of the methods described herein.

In another aspect, this disclosure features a kit for use in a method of detecting analyte in a biological sample, wherein the kit includes any two or more of: (a) a first probe oligonucleotide and a second probe oligonucleotide, wherein the first probe oligonucleotide and the second probe oligonucleotide are substantially complementary to adjacent sequences of the analyte, and wherein the second probe oligonucleotide includes a 5′ FLAP; (b) an endonuclease, wherein the endonuclease cleaves the 5′ FLAP thereby releasing the 5′ FLAP from the second probe oligonucleotide; and (c) instructions for carrying out any of the methods described herein.

In another aspect, this disclosure features a kit for use in a method of detecting a genetic variant in an gDNA molecule in a biological sample, wherein the kit includes any two or more of: (a) a first probe oligonucleotide and a second probe oligonucleotide, wherein the first probe oligonucleotide and the second probe oligonucleotide each include a sequence that is substantially complementary to adjacent sequences of the gDNA molecule; wherein the first probe oligonucleotide and the second probe oligonucleotide are capable of forming an invasive cleavage structure in the presence of the genetic variant; and wherein the second probe oligonucleotide further includes a 5′ FLAP; (b) an endonuclease, wherein the endonuclease cleaves the 5′ FLAP thereby releasing the 5′ FLAP from the second probe oligonucleotide; (c) a substrate including a plurality of capture probes, wherein a capture probe of the plurality includes a spatial barcode and a capture domain, wherein the 5′ FLAP is capable of hybridizing to the capture domain; and (d) instructions for carrying out any of the methods described herein.

In another aspect, this disclosure features a kit for use in a method of detecting a genetic variant in an gDNA molecule in a biological sample, wherein the kit includes any two or more of: (a) a first probe oligonucleotide and a second probe oligonucleotide, wherein the first probe oligonucleotide and the second probe oligonucleotide each include a sequence that is substantially complementary to adjacent sequences of the gDNA molecule; wherein the first probe oligonucleotide and the second probe oligonucleotide are capable of forming an invasive cleavage structure in the presence of the genetic variant; and wherein the second probe oligonucleotide further includes a 5′ FLAP; (b) an endonuclease, wherein the endonuclease cleaves the 5′ FLAP thereby releasing the 5′ FLAP from the second probe oligonucleotide; and (c) instructions for carrying out any of the methods described herein.

In another aspect, this disclosure features compositions including: a first probe oligonucleotide and a second probe oligonucleotide hybridized to an analyte, wherein the first probe oligonucleotide and the second probe oligonucleotide are substantially complementary to adjacent sequences of the analyte, and wherein the second probe oligonucleotide includes a 5′ FLAP.

In another aspect, this disclosure features compositions including: a first probe oligonucleotide and a second probe oligonucleotide hybridized to an analyte, wherein the first probe oligonucleotide and the second probe oligonucleotide each include a sequence that is substantially complementary to adjacent sequences of the gDNA molecule; wherein the first probe oligonucleotide and the second probe oligonucleotide are capable of forming an invasive cleavage structure in the presence of the genetic variant; and wherein the second probe oligonucleotide further includes a 5′ FLAP. In some embodiments, the 5′ FLAP includes a sequence that is capable of hybridizing to a capture domain of a capture probe. In some embodiments, the capture probe further includes a spatial barcode. In some embodiments, the capture probe is part of a plurality of capture probes affixed to a substrate.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a cell and bind to target analytes within the sample.

FIG. 3 is a schematic diagram of an exemplary multiplexed spatially-barcoded feature.

FIG. 4 is a schematic showing the arrangement of barcoded features within an array.

FIG. 5 shows a schematic of an example workflow for using barcoded 5′ FLAP probes for the detection of a gDNA analyte. LHS: left hand templated ligation probe; RHS; right hand templated ligation probe; smRNA R2 handle: primer.

FIG. 6 shows a schematic of an example workflow for using barcoded 5′ FLAP probes to detect a genetic variant in a gDNA analyte. 1nt: one nucleotide.

FIG. 7 shows a schematic of an example workflow for using barcoded 5′ FLAP probes for detecting a gDNA analyte at a spatial location in a biological sample.

FIG. 8 shows a schematic of an example workflow for using barcoded 5′ FLAP probes for detecting a genetic variant in a gDNA analyte at a spatial location in a biological sample

FIGS. 9A and 9B show a schematic of an example workflow where a barcoded 5′ FLAP probes is specifically binds to a capture probe on a substrate.

### DETAILED DESCRIPTION

### I. Methods and Compositions for Spatial Analysis

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, the capture probe further includes a unique molecular identifier. In some embodiments, the capture probe further includes a functional domain. In some embodiments, the capture domain includes a sequence that is partially complementary to the analyte or an analyte binding moiety. In some embodiments, the capture domain includes a homopolymeric sequence. In some embodiments, the capture domain includes a poly(T) sequence. In some embodiments, the capture probe further includes a cleavage domain. In some embodiments, the cleavage domain includes a cleavable linker. Non-limiting examples of a cleavable linker include a photocleavable linker, a UV-cleavable linker, an enzymatic cleavable linker, or a pH-sensitive cleavable linker. In some embodiments, the plurality of capture probes are attached to one or more features.

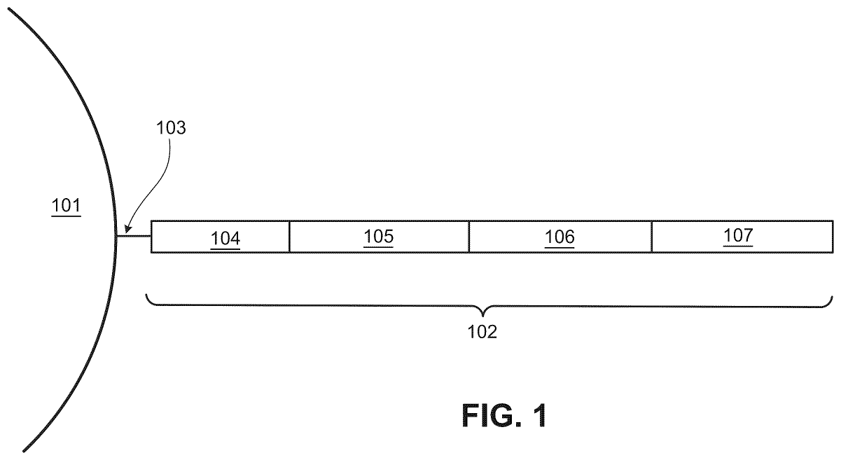

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a ligation product described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence complementary to a sequence of a nucleic acid analyte, a portion of a ligation product described herein, a capture handle sequence described herein, and/or a methylated adaptor described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

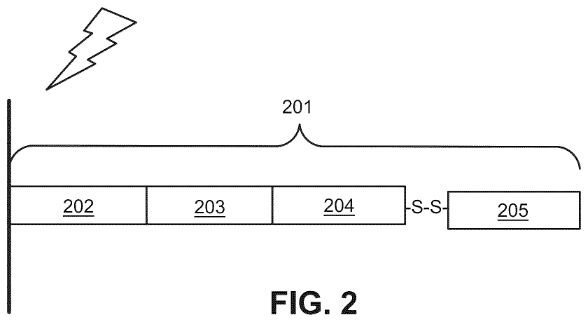

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain. Cleavable capture probe are further described in WO 2020/176788 and/or U.S. Patent Application Publication No. 2020/0277663, each of which is incorporated by reference in its entirety.

For multiple capture probes that are attached to a common array feature, the one or more spatial barcode sequences of the multiple capture probes can include sequences that are the same for all capture probes coupled to the feature, and/or sequences that are different across all capture probes coupled to the feature.

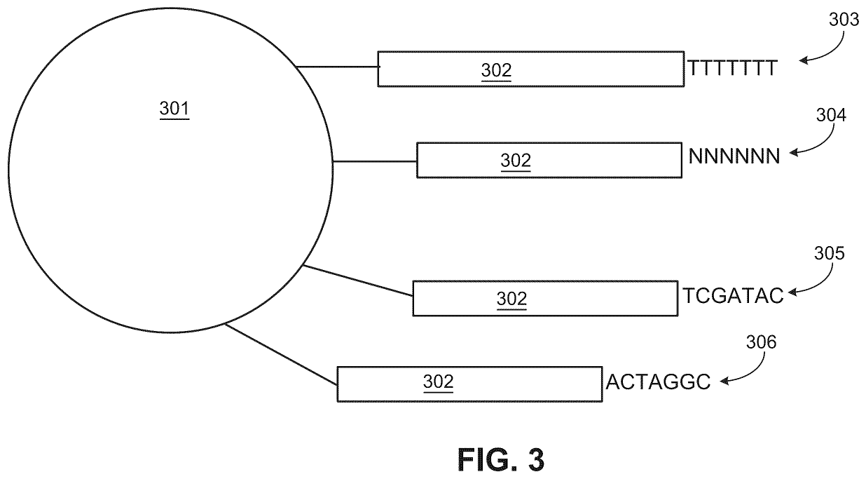

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

Capture probes attached to a single array feature can include identical (or common) spatial barcode sequences, different spatial barcode sequences, or a combination of both. Capture probes attached to a feature can include multiple sets of capture probes. Capture probes of a given set can include identical spatial barcode sequences. The identical spatial barcode sequences can be different from spatial barcode sequences of capture probes of another set.

The plurality of capture probes can include spatial barcode sequences (e.g., nucleic acid barcode sequences) that are associated with specific locations on a spatial array. For example, a first plurality of capture probes can be associated with a first region, based on a spatial barcode sequence common to the capture probes within the first region, and a second plurality of capture probes can be associated with a second region, based on a spatial barcode sequence common to the capture probes within the second region. The second region may or may not be associated with the first region. Additional pluralities of capture probes can be associated with spatial barcode sequences common to the capture probes within other regions. In some embodiments, the spatial barcode sequences can be the same across a plurality of capture probe molecules.

In some embodiments, multiple different spatial barcodes are incorporated into a single arrayed capture probe. For example, a mixed but known set of spatial barcode sequences can provide a stronger address or attribution of the spatial barcodes to a given spot or location, by providing duplicate or independent confirmation of the identity of the location. In some embodiments, the multiple spatial barcodes represent increasing specificity of the location of the particular array point.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence or capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

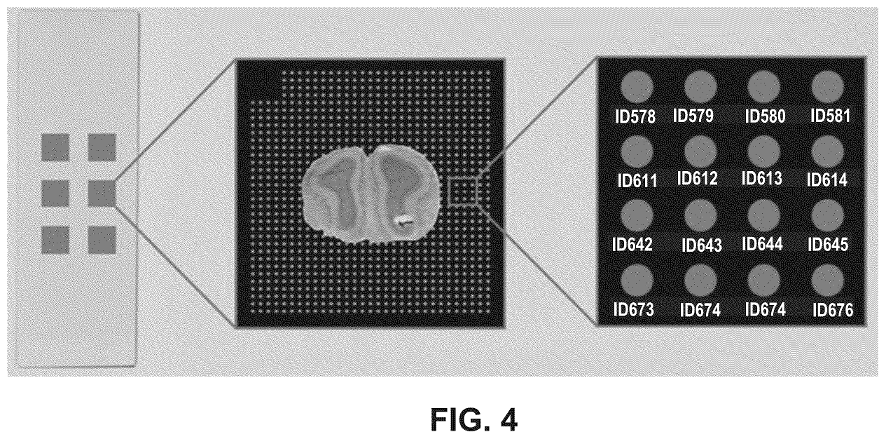

FIG. 4 depicts an exemplary arrangement of barcoded features within an array. From left to right, FIG. 4 shows (L) a slide including six spatially-barcoded arrays, (C) an enlarged schematic of one of the six spatially-barcoded arrays, showing a grid of barcoded features in relation to a biological sample, and (R) an enlarged schematic of one section of an array, showing the specific identification of multiple features within the array (labelled as ID578, ID579, ID560, etc.).

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion).

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14): e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distance.

II. Using Barcoded 5′ FLAP Probes for the Detection of gDNA

### (a) DNA-Templated Ligation Using a 5′ FLAP Sequence

Templated ligation, or RNA templated ligation (RTL), is a process that includes multiple RTL probes (e.g., usually two oligonucleotides or probes) that hybridize to adjacent complementary mRNA sequences. RNA-templated ligation is enabled by the ability of RNases to remove the RNA in a RNA-DNA hybrid. Provided herein are methods that co-opt RTL principles to include oligonucleotide probes that hybridize to adjacent complementary genomic DNA (gDNA) sequences. It is known that DNA-DNA hybrids are harder to separate than RNA-DNA hybrids. Therefore, a readout of the DNA-templated ligation event is needed that can be measured without having to make single stranded DNA from the DNA-DNA hybrid. Here, an oligonucleotide (e.g., a second RTL probe) includes a 5′ FLAP sequence that can be removed and detected following an enzyme-mediated cleavage event that does not require removal of the hybridized probes. Detection of the 5′ FLAP provides a readout for the DNA-templated ligation event, which can serve as a proxy for detecting a gDNA analyte or a genetic variant within a gDNA analyte.

The methods provided herein rely on the cleavage of the 5′ FLAP from the second RTL probe occurring when the first RTL probe and the second RTL probe hybridize to a gDNA analyte at adjacent sequences. In some cases, the adjacent sequences flank a target sequence (e.g., a genetic variant). In some cases, the adjacent sequences directly abut each other in the gDNA analyte. Once a first RTL probe and a second RTL probe hybridize to a gDNA analyte, an enzyme-mediated cleavage event cleaves the second RTL probe, which includes non-complementary nucleotides (e.g., a 5′ FLAP). Upon cleavage of the 5′ FLAP, the sequence of the 5′ FLAP can be determined and used to detect the presence of a gDNA analyte in a biological sample or the presence of a genetic variant in a gDNA analyte. In some instances, determining the sequence of the 5′ FLAP includes hybridizing the 5′ FLAP to a probe on an array. In such cases, the 5′ FLAP includes a capture probe binding domain sequence (e.g., a poly-adenylation sequence or a target nucleic acid sequence) that can be detected by a capture probe on an array described herein (e.g., the capture probe comprises a poly-thymine sequence in some instances). In some instances, determining the sequence of the 5′ FLAP includes sequencing the 5′ FLAP without first hybridizing the 5′ FLAP to a capture probe on an array.

Thus, the methods provided herein allow detection of a gDNA analyte or a genetic variant in a gDNA analyte and in some cases enable detection of the spatial location within a biological sample. In addition, the methods provided herein allow detection of a gDNA analyte within a subset of DNA analytes, thereby increasing the resolution of the technology as applied to gDNA. Also, the methods provided herein avoid the need for probe ligation and endoribonuclease (e.g., RNaseH) treatment, which are both used in a standard RTL workflow, thereby keeping costs low without sacrificing efficiency.

Disclosed herein are methods for detecting an gDNA analyte in a biological sample where (i) the first RTL probe and second RTL probe hybridize to adjacent sequences on the gDNA analyte, (ii) enzyme-mediated cleavage of a 5′ FLAP results in release of the 5′ FLAP, and (iii) the sequence of the 5′ FLAP is determined and used to detect the gDNA in the biological sample. In some embodiments, the method includes detecting a gDNA analyte at a spatial location in a biological sample using a substrate to capture the released 5′ FLAP. In such cases, the method includes contacting the biological sample with a substrate that has a plurality of capture probes, where a capture probe of the plurality of capture probes includes a spatial barcode and a capture domain, prior to the RTL probe hybridization step. After cleavage of the 5′ FLAP, it hybridizes to the capture domain, and the sequence of the 5′ FLAP is determined. The along with determination of the spatial barcode, the spatial location of the gDNA analyte in the biological sample can be detected.

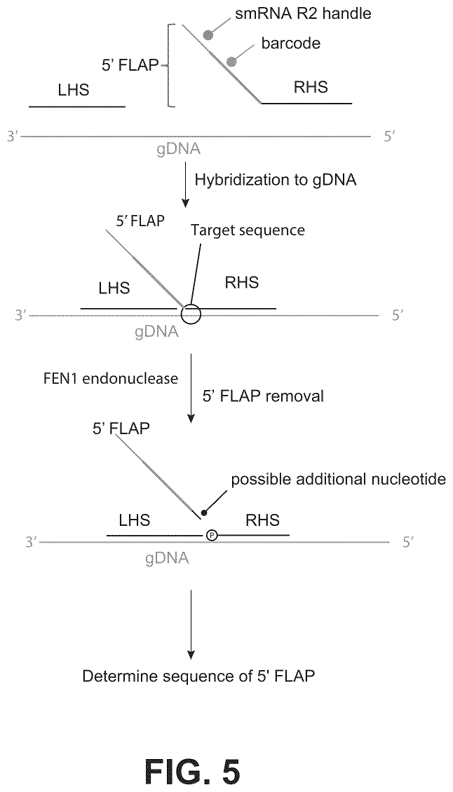

The methods disclosed herein use compositions comprising an RTL probe (e.g., a second RTL probe) that has a non-hybridizing sequence that can be used to carry information about the hybridization event between an analyte and an oligonucleotide. In some instances, the non-hybridizing sequence is referred to as a 5′ FLAP. See e.g., FIG. 5, which shows the non-hybridizing 5′ FLAP on the Right Hybridizing Sequence (RHS) RTL probe. As disclosed herein, the sequence of the 5′ FLAP is used to convey information about a gDNA analyte. For example, described herein are methods were the presence of a 5′ FLAP sequence in a sequencing library indicates either the presence or absence of a gDNA analyte or the presence or absence of a genetic variant in an gDNA analyte. In some embodiments, the methods provided herein further include contacting a biological sample that includes the 5′ FLAP with a substrate in order to determine the location of the gDNA analyte or genetic variant within the biological sample. In some instances, the 5′ FLAP can convey information about a gDNA analyte via other mechanisms including by labeling the 5′ FLAP with a detectable label (e.g., any of the exemplary detectable labels described herein) and then detecting the label and using detection of the label as a proxy for the presence or absence of a gDNA analyte or genetic variant in a gDNA analyte.

In some embodiments, the subset of analytes includes gDNA analytes including DNA that is transcribed into RNA (e.g., coding regions including, without limitation, gene exons, gene introns, and untranslated regions (both 5′ and 3′)) and DNA that is not transcribed into RNA but regulates transcription of DNA into RNA (e.g., regulatory sequences including, without limitation, promoters, enhancers, and silencers). Additional gDNA analytes include gDNA sequences associated with a particular region of a chromosome (e.g., without limitation, a telomere, a centromere, a topologically associated domain, an arm of a chromosome, a portion of an arm of a chromosome, a condensed chromosome, and an uncondensed chromosome).

In some embodiments, the subset of analytes includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700, about 800, about 900, about 1000, or more analytes.

Methods disclosed herein can be performed on any type of sample. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample. FFPE samples generally are heavily cross-linked and fragmented, and therefore this type of sample allows for limited DNA recovery using conventional detection techniques. In certain embodiments, methods of targeted DNA capture provided herein are less affected by DNA degradation associated with FFPE fixation than other methods (e.g., methods that take advantage of oligo-dT capture and reverse transcription of gDNA). In certain embodiments, methods provided herein enable sensitive measurement of specific genes of interest that otherwise might be missed with a whole transcriptomic approach.

In some embodiments, a biological sample (e.g. tissue section) can be fixed with methanol, stained with hematoxylin and eosin, and imaged. In some embodiments, fixing, staining, and imaging occurs before one or more RTL probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the methods of targeted DNA capture as disclosed herein include hybridization of multiple RTL probes. In some embodiments, the methods include 2, 3, 4, or more RTL probes that hybridize to one or more analytes of interest. In some embodiments, the methods include two RTL probes. In some embodiments, the RTL probe includes sequences complementary that are complementary or substantially complementary to an analyte. For example, in some embodiments, the RTL probe includes a sequence that is complementary or substantially complementary to an analyte (e.g., a gDNA of interest (e.g., to a portion of the sequence of a gDNA of interest)). Methods provided herein may be applied to a single nucleic acid molecule or a plurality of nucleic acid molecules. A method of analyzing a sample comprising a nucleic acid molecule may comprise providing a plurality of nucleic acid molecules (e.g., DNA molecules), where each nucleic acid molecule comprises a first target region (e.g., a sequence that is 3′ of a target sequence or a sequence that is 5′ of a target sequence) and a second target region (e.g., a sequence that is 5′ of a target sequence or a sequence that is 3′ of a target sequence), a plurality of first RTL probes, and a plurality of second RTL probes.

After hybridization of the first and second RTL probes, the first RTL probe can be extended. After hybridization, in some embodiments, the second RTL probe can be extended. Extending probes can be accomplished using any method disclosed herein. In some instances, a polymerase (e.g., a DNA polymerase) extends the first and/or second RTL probe.

In some embodiments, methods disclosed herein include a wash step. In some instances, the wash step occurs after hybridizing the first and the second RTL probes. The wash step removes any unbound probes and can be performed using any technique or solution disclosed herein or known in the art. In some embodiments, multiple wash steps are performed to remove unbound RTL probes.

In some embodiments, after hybridization of RTL probes (e.g., first and the second RTL probes) to the gDNA analyte, the 5′ FLAP is cleaved, releasing the 5′ FLAP. Releasing the 5′ FLAP can be performed enzymatically as described herein. As disclosed in the following sections, releasing the 5′ FLAP allows for detection of a gDNA analyte of interest.

(i) gDNA Detection in a Biological Sample Using a 5′ FLAP

This disclosure features a method for detecting a gDNA analyte in a biological sample using methods that co-opt RTL principles to include RTL probes that hybridize to adjacent complementary genomic DNA (gDNA) sequences. In particular, the methods disclosed herein utilize a process that cleaves a non-hybridized sequence of one of the RTL probes. The non-hybridized sequence (e.g., a 5′ FLAP) can be further analyzed to determine the presence or absence of a gDNA analyte of interest.

A non-limiting example of a method for detecting a gDNA analyte in a biological sample includes: (a) contacting the biological sample with a first RTL probe and a second RTL probe, wherein the first RTL probe and the second RTL probe are substantially complementary to adjacent sequences of the gDNA analyte, and wherein the second RTL probe includes a 5′ FLAP; (b) hybridizing the first RTL probe and the second RTL probe to the gDNA analyte; (c) cleaving the second RTL probe, thereby releasing the 5′ FLAP; (d) determining all or a part of the sequence of the 5′ FLAP, and using the sequence of the 5′ FLAP to detect the gDNA analyte. In some embodiments, the method also includes denaturing the gDNA under conditions wherein the first RTL probe and the second RTL probe can hybridize to the gDNA analyte. In some embodiments, the method includes a denaturing step prior to contacting the biological sample with a first RTL probe and a second RTL probe. In some embodiments, the method further includes, prior to (a), contacting the biological sample with a substrate comprising capture probes. In some embodiments, the method further includes contacting the biological sample with a permeabilization reagent. In some embodiments, the biological sample is contacted with the permeabilization reagent before determining all or a part of the sequence of the 5′ FLAP, and using the sequence of the 5′ FLAP to detect the gDNA analyte. In some embodiments, the method also includes determining step includes amplifying all or part of the 5′ FLAP. In some embodiments, the amplifying includes isothermal amplification. In some embodiments, the determining step includes sequencing.

In some embodiments, the cleaving step includes contacting with an endonuclease. The endonuclease can include a 5′ FLAP endonuclease activity where the enzyme catalyzes the cleavage of a 5′ FLAP structure. The step uses two RTL probes that are hybridized to target nucleic acid (e.g., gDNA) containing, for example, a polymorphic site. One RTL probe is complementary to the target sequence 3′ of the polymorphic site and ends with a non-matching base overlapping the SNP nucleotide. The second RTL probe, the allele-specific probe, contains the complementary base of the SNP allele and extends to the sequence 5′ of the polymorphic site. This probe can also extend on its 5′ site with additional non-complementary nucleotides. An invasive cleavage structure is formed when the first RTL probe and the second RTL probe are both hybridized to the gDNA molecule. Once the two oligonucleotides anneal to the target DNA, they form a three-dimensional invasive structure over the SNP site that can be recognized by cleavase, a FEN enzyme. The enzyme cleaves the probe 3′ of the base complementary to the polymorphic site (i.e. 3′ of the overlapping invader structure). After hybridization, the invasive cleavage structure is formed when the first RTL probe and the second RTL probe are both hybridized to the gDNA analyte. Additional exemplary support for the invasive cleavage structure utilized in an Invader assay is provided in Olivier, Mutat. Res., 2005 Jun. 3; 573(1-2): 103-110, which is incorporated by reference in its entirety.

In some embodiments, the endonuclease cleaves a portion of the second RTL probe. In some embodiments, the endonuclease cleaves the 5′ FLAP of the second RTL probe. Non-limiting examples of endonucleases with 5′-FLAP endonuclease activity that can be used in the cleaving step include DNA replication helicase 2 (DNA2), Exonuclease 1 (EXO1), FANCD2/FANCI-associated nuclease 1 (FAN1), Holliday junction 5′ flap endonuclease (GEN1), structure-specific endonuclease subunit homolog B (SLX1B), and GRF-type containing 1 (ZGRF1). In some embodiments, the endonuclease is FLAP endonuclease 1 (FEN1).

In some embodiments, the cleaving step includes providing a DNA-dependent DNA polymerase. The DNA-dependent DNA polymerase can include 3′ to 5′ exonuclease activity. The DNA polymerase can include strand displacement properties. For example, the DNA polymerase uses the strand displacement property to displace the second RTL probe and an endonuclease cleaves the portion of the second RTL probe not bound (e.g., the 5′ FLAP and the portion of the second RTL probe displaced by the DNA polymerase) to the target sequence or the sequence 5′ of the target sequence. The portion of the second RTL probe not bound to the target sequence or the sequence 5′ of the target sequence includes the 5′ FLAP. Non-limiting examples of a DNA polymerase that can be used in the cleaving step include Phi29 DNA polymerase and Taq DNA polymerase.

In some embodiments, the 5′ FLAP further includes a first barcode sequence. In some embodiments, the first barcode sequence includes a sequence that identifies the first RTL probe, the second RTL probe, and/or the gDNA analyte. In some instances, the first barcode sequence is a sequence unique to the interaction between the first RTL probe, the second RTL probe, and/or the gDNA analyte. Thus, it can be decoded to determine the presence of the interaction between the first RTL probe, the second RTL probe, and/or the gDNA analyte. In some instances, the first barcode sequence can be used to identify the second RTL probe. When the 5′ FLAP is cleaved, the 5′ FLAP can be used to identify a hybridization event where both the first RTL probe and the second RTL probe hybridize to a gDNA analyte.

In some embodiments, the 5′ FLAP also includes a functional sequence. The functional sequence can be a primer sequence. The primer sequence can be used to amplify the 5′ FLAP before cleavage, contemporaneously with cleavage, or after cleavage of the 5′ FLAP from the second RTL probe.

In some embodiments, the second RTL probe includes a second barcode. For example, the second barcode is on the portion of the second RTL probe that is not released following cleavage. In some embodiments, the second barcode can be used to identify the second RTL probe.

This disclosure features a method for detecting a gDNA analyte at a spatial location in a biological sample using methods that co-opt RTL principles to include RTL probes that hybridize to adjacent complementary genomic DNA (gDNA) sequences. In particular, the methods disclosed herein utilizes a process that cleaves a non-hybridized sequence of an RTL probe. The non-hybridized sequence (e.g., a 5′ FLAP) can be further analyzed to determine the presence or absence of a gDNA analyte of interest.

In some instances, a method for detecting a gDNA analyte at a spatial location in a biological sample includes: (a) contacting the biological sample with a substrate including a plurality of capture probes, wherein a capture probe of the plurality includes a spatial barcode and a capture domain; (b) contacting a biological sample with a first RTL probe and a second RTL probe, wherein the first RTL probe and the second RTL probe are substantially complementary to adjacent sequences of the gDNA analyte, and wherein the second RTL probe includes a 5′ FLAP; (c) hybridizing the first RTL probe and the second RTL probe to the gDNA analyte; (d) cleaving the second RTL probe, thereby releasing the 5′ FLAP; (e) hybridizing the 5′ FLAP to the capture domain; and (f) determining (i) all or a part of the sequence of the 5′ FLAP, or a complement thereof, and (ii) all or a part of the sequence of the spatial barcode, or a complement thereof, and using the determined sequence of (i) and (ii) to identify the location of the gDNA analyte in the biological sample. In some embodiments, the method also includes denaturing the gDNA under conditions wherein the first RTL probe and the second RTL probe can hybridize to the gDNA analyte. In some embodiments, the method includes a denaturing step prior to contacting a biological sample with a first RTL probe and a second RTL probe. In some embodiments, the method further includes contacting the biological sample with a permeabilization reagent. In some embodiments, the biological sample is contacted with the permeabilization reagent before contacting the biological sample with a substrate including a plurality of capture probes. In some embodiments where the method includes detecting an analyte at a spatial location in a biological sample, the determining step includes amplifying all or part of the 5′ FLAP specifically bound to the capture domain. For example, the amplification can be isothermal. The determining step can include sequencing the 5′ FLAP.

In some embodiments, the cleaving step includes providing an endonuclease. The endonuclease can include a 5′ FLAP endonuclease activity where the enzyme catalyzes the cleavage of a 5′ FLAP structure. In some embodiments, the endonuclease cleaves a portion of second RTL probe. In some embodiments, the endonuclease cleaves the 5′ FLAP of the second RTL probe. Non-limiting examples of endonucleases with 5′-FLAP endonuclease activity that can be used in the cleaving step include DNA replication helicase 2 (DNA2), Exonuclease 1 (EXO1), FANCD2/FANCI-associated nuclease 1 (FAN1), Holliday junction 5′ flap endonuclease (GEN1), structure-specific endonuclease subunit homolog B (SLX1B), and GRF-type containing 1 (ZGRF1). In some embodiments, the endonuclease is FLAP endonuclease 1 (FEN1).

In some embodiments, the cleaving step includes providing a DNA-dependent DNA polymerase. The DNA-dependent DNA polymerase can include 3′ to 5′ exonuclease activity. The DNA polymerase can include strand displacement properties. For example, the DNA polymerase uses the strand displacement property to displace the second RTL probe and an endonuclease cleaves the portion of the second RTL probe not bound to the target sequence (e.g., the 5′ FLAP and the portion of the second RTL probe displaced by the DNA polymerase) or the sequence 5′ of the target sequence. The portion of the second RTL probe not bound to the target sequence or the sequence 5′ of the target sequence includes the 5′ FLAP. Non-limiting examples of DNA polymerase that can be used in the cleaving step include Phi29 DNA polymerase and Taq DNA polymerase.

In some embodiments, the method also includes providing a capture probe binding domain blocking moiety that interacts with the capture probe binding domain. In some embodiments, the method further includes releasing the capture probe binding domain blocking moiety from the capture probe binding domain prior to contacting the biological sample with the substrate. In some embodiments, the capture probe binding domain blocking moiety includes a poly-uridine sequence, a poly-thymidine sequence, or both. In some embodiments, releasing the capture probe binding domain blocking moiety from the poly(A) sequence includes denaturing the 5′ FLAP.

In some embodiments, the method for detecting an analyte at a spatial location in a biological sample includes a second RTL probe having a 5′ FLAP that further includes a first barcode sequence. In some embodiments, the first barcode sequence includes a sequence that identifies the first RTL probe, the second RTL probe, and/or the gDNA analyte. In some embodiments, the 5′FLAP further includes a functional sequence. For example, the functional sequence is a primer sequence.

In some embodiments, the 5′ FLAP further includes a capture probe binding domain. The capture domain includes a sequence that is substantially complementary to a capture domain on a capture probe where the capture probe binding domain sequence enables binding of the 5′ FLAP to the capture probe. The capture probe binding domain can include a homopolymeric sequence. For example, without limitation, the capture probe binding domain can be a poly(A) sequence. In some embodiments, the method for detecting an analyte at a spatial location in a biological sample includes a second RTL probe that includes from 5′ to 3′: a 5′ FLAP and a sequence that is substantially complementary to a sequence that is adjacent to a sequence that is substantially complementary to a first RTL probe. In other cases, the second RTL probe includes a 5′ FLAP including from 5′ to 3′: a functional sequence, a first barcode, and a capture probe binding domain sequence.

In some embodiments, the second RTL probe further includes a second barcode. In some embodiments, the second barcode sequence includes a sequence that identifies the first RTL probe, the second RTL probe, and/or the gDNA analyte. In some embodiments, the second barcode sequence includes a sequence that identifies the second RTL probe. A second RTL probe can include from 5′ to 3′: a 5′ FLAP, a sequence that is substantially complementary to a sequence that is adjacent to a sequence that is substantially complementary to a first RTL probe, and a second barcode.

In some embodiments, the second RTL probe further includes a second capture probe binding domain. For example, a second RTL probe can include form 5′ to 3′: a 5′ FLAP, a sequence that is substantially complementary to a sequence that is adjacent to a sequence that is substantially complementary to a first RTL probe, a second barcode, and a second capture probe binding domain. The second capture domain can include any of the exemplary capture probe binding domain sequences describe herein. The second capture probe binding domain enables the portion of the second RTL probe that is not cleaved to hybridize to a capture domain on a capture probe.

### (b) SNP Detection Using Templated Ligation and Detection of the 5′ FLAP

This disclosure also features methods of detecting a SNP using two oligonucleotide probes that are hybridized to a gDNA analyte containing a polymorphic site. The two oligonucleotides hybridize to the single-stranded target and form an overlapping invader structure at the site of the SNP. One oligonucleotide (e.g., a first RTL probe) is complementary to the target sequence 3′ of the polymorphic site and ends with a non-matching base overlapping the SNP nucleotide. The second oligonucleotide (e.g., the second RTL probe), the allele-specific probe, contains the complementary base of the SNP allele and extends to the sequence 5′ of the polymorphic site. In some instances, this second oligonucleotide can also include a 5′ FLAP including additional non-complementary nucleotides (e.g., a functional sequence and a barcode sequence). Once the two oligonucleotides hybridize to the target DNA, they form a three-dimensional invader structure over the SNP site that can be recognized by a cleavase, such as a FEN enzyme. An invasive cleavage structure is described in U.S. Pat. No. 6,913,881 B1, which is incorporated by reference in its entirety. The enzyme cleaves the 5′ FLAP (e.g., the non-complementary nucleotides) of the second RTL probe. Once cleaved, 5′ FLAP is released. Additional exemplary support for the invasive cleavage structure utilized in an Invader assay is provided in Olivier, Mutat. Res. 2005 Jun. 3; 573(1-2): 103-110, which is incorporated by reference in its entirety. In an alternative embodiment, the cleaved 5′ FLAP can be detected using a FRET pair, wherein the release of the FLAP results in fluorescence detection. In some instances, the 5′ FLAP includes a tag (e.g., a fluorescent tag) that can be detected upon cleavage. In another embodiment, after cleavage, the supernatant can be extracted and sequencing, providing the ability to detect the presence of a SNP or mutation of interest.

In some embodiments, a method for detecting a gDNA analyte in a biological sample includes (i) hybridizing a first RTL probe and a second RTL probe to adjacent sequences on the gDNA analyte, (ii) cleaving a 5′ FLAP from the second RTL probe, thereby releasing the 5′ FLAP, and (iii) determining the sequence of the 5′ FLAP and using the sequence of the 5′ FLAP to detect a genetic variant in the gDNA in the biological sample. In some embodiments, the method includes detecting a gDNA analyte at a spatial location in a biological sample using a substrate to capture the released 5′ FLAP. In such cases, the method further includes contacting the biological sample with a substrate including a plurality of capture probes, wherein, a capture probe of the plurality includes a spatial barcode and a capture domain, hybridizing the 5′ FLAP to the capture domain, and determining the sequence of the 5′ FLAP and using the sequence of the 5′ FLAP to detect a genetic variant in a gDNA analyte at a spatial location in the biological sample.

In some embodiments, a method of detecting a genetic variant in a gDNA analyte in a biological sample includes determining all or a part of the sequence of the 5′ FLAP, and using the sequence of the 5′ FLAP to detect the genetic variant in the gDNA analyte without using a substrate including capture probes to capture the 5′ FLAP. In such cases, the 5′ FLAP does not include a capture probe binding domain sequence. In such cases, a gDNA analyte is contacted with a first RTL probe and a second RTL probe, wherein the first RTL probe and the second RTL probe each include a sequence that is substantially complementary to adjacent sequences of the gDNA analyte; wherein the first RTL probe and the second RTL probe are capable of forming an invasive cleavage structure in the presence of the genetic variant; and wherein the second RTL probe further includes a 5′ FLAP. The 5′ FLAP can include a barcode sequence that can be used to identify the first RTL probe, the second RTL probe, and/or the gDNA analyte. In some cases, the method also includes denaturing the gDNA under conditions wherein the first RTL probe and the second RTL probe can hybridize to the gDNA analyte. The first RTL probe and the second RTL probe can hybridize to the gDNA analyte. Following hybridization in the presence of the genetic variant, the second RTL probe can be cleaved, thereby releasing the 5′ FLAP. Finally, all or a part of the sequence of the 5′ FLAP is determined, and used to detect the genetic variant in the gDNA analyte.

In another embodiment, this disclosure features a method of detecting a genetic variant in a gDNA analyte at a spatial location in a biological sample. In a non-limiting example this method includes contacting the biological sample with a first RTL probe and a second RTL probe, wherein the first RTL probe and the second RTL probe each include a sequence that is substantially complementary to adjacent sequences of the gDNA analyte; wherein the first RTL probe and the second RTL probe are capable of forming an invasive cleavage structure when the genetic variant is present; and wherein the second RTL probe further includes a 5′ FLAP. The 5′ FLAP can include a barcode sequence that can be used to identify the first RTL probe, the second RTL probe, and/or the gDNA analyte and a capture probe binding domain sequence that can be used to hybridize the 5′ FLAP to capture domain of a capture probe located on the surface of a substrate. In some cases, the method also includes denaturing the gDNA under conditions wherein the first RTL probe and the second RTL probe can hybridize to the gDNA analyte. Following hybridization of the first RTL probe and the second RTL probe in the presence of the genetic variant, the second RTL probe can be cleaved, thereby releasing the 5′ FLAP. The method then includes contacting the biological sample with a substrate including a plurality of capture probes, wherein a capture probe of the plurality includes a spatial barcode and a capture domain; hybridizing the 5′ FLAP to the capture domain; and determining (i) all or a part of the sequence of the 5′ FLAP, or a complement thereof, and (ii) all or a part of the sequence of the spatial barcode, or a complement thereof, and using the determined sequence of (i) and (ii) to identify the genetic variant in the gDNA analyte at a spatial location in the biological sample.

In some embodiments, the first RTL probe includes a sequence that is substantially complementary to a sequence 3′ of the target sequence (e.g., the genetic variant). In some embodiments, the first RTL probe is at least 70% identical (e.g., at least 75% identical, at least 80% identical, at least 85% identical, at least 90% identical, at least 95% identical, or at least 99% identical) to a sequence 3′ of the target sequence (e.g., the genetic variant).

In some embodiments, the first RTL probe includes a sequence that is substantially complementary to a sequence 5′ of the target sequence (e.g., the genetic variant). In some embodiments, the first RTL probe is at least 70% identical (e.g., at least 75% identical, at least 80% identical, at least 85% identical, at least 90% identical, at least 95% identical, or at least 99% identical) to a sequence 5′ of the target sequence (e.g., the genetic variant).

In some embodiments, the first RTL probe includes at least one nucleotide that is complementary to a reference target sequence (e.g., wild type sequence of a genetic variant). In some embodiments, the first RTL probe includes at least one nucleotide that is complementary to a single nucleotide polymorphism (e.g., a single nucleotide polymorphism as compared to a reference target sequence).

In some embodiments, the second RTL probe includes a sequence substantially complementary to a sequence 5′ to the target sequence (e.g., the genetic variant). In some embodiments, the second RTL probe is at least 70% identical (e.g., at least 75% identical, at least 80% identical, at least 85% identical, at least 90% identical, at least 95% identical, or at least 99% identical) to a sequence 5′ of the target sequence (e.g., the genetic variant).

In some embodiments, the second RTL probe includes a sequence substantially complementary to a sequence 3′ to the target sequence (e.g., the genetic variant). In some embodiments, the second RTL probe is at least 70% identical (e.g., at least 75% identical, at least 80% identical, at least 85% identical, at least 90% identical, at least 95% identical, or at least 99% identical) to a sequence 3′ of the target sequence (e.g., the genetic variant).

In some embodiments, the second RTL probe includes at least one nucleotide complementary to the at least one genetic variant of the target sequence. In some embodiments, the second RTL probe includes at least one nucleotide complementary to a reference target sequence (e.g., a wild type sequence).

In some embodiments, the second RTL probe includes from 5′ to 3′: a sequence of non-complementary nucleotides (e.g., a 5′ FLAP), one or more nucleotides complementary to the target sequence (e.g., the genetic variant), and a sequence substantially complementary to a sequence 5′ of the target sequence (e.g., the genetic variant). In such cases, the 5′ FLAP includes a barcode sequence. Where the 5′ FLAP sequence is determined without hybridizing the 5′ FLAP to a capture probe, the 5′ FLAP does not include a capture probe binding domain sequence. In some cases where the 5′ FLAP sequence is determined without hybridizing the 5′ FLAP to a capture probe, the 5′ FLAP includes a capture probe binding domain sequence. In cases where the sequence of the 5′ FLAP is determined by hybridizing the 5′ FLAP to a capture probe (i.e., via a capture domain), the 5′ FLAP includes a capture probe binding domain sequence.

In some embodiments, the target sequence in an analyte of interest includes one or more nucleotides. In some embodiments, the target sequence includes one or more single nucleotide variants (SNV) compared to a reference target sequence. In some instances, the target sequence includes one SNV compared to a reference target sequence. In some embodiments, the at least one genetic variant is a single nucleotide polymorphism (SNP). For example, a gDNA analyte can include one or more SNPs compared to a reference gDNA analyte. In some embodiments, the at least one genetic variant is a nucleotide point mutation. In some embodiments, the at least one genetic variant includes at least two, at least three, at least four, at least five, or more genetic variants.

In some embodiments, after formation of the invader structure, the biological sample is contacted with an endonuclease. In some instances, the endonuclease cleaves the sequence of non-complementary nucleotides (e.g. a 5′ FLAP) of the second RTL probe. In some embodiments, the endonuclease cleaves a portion of the first RTL probe. In some embodiments, the endonuclease is FLAP endonuclease 1 (FEN1). FEN1 is a structure-specific endonuclease that cuts at the base of single-stranded FLAPs. See Balakrishnan and Bambara, Annu Rev Biochem. 2013 Jun. 2; 82: 119-138, which is incorporated by reference in its entirety. In some embodiments, the endonuclease cleaves the at least one nucleotide that is complementary to a wild-type sequence of the target sequence of the first RTL probe.

In some instances, once FEN1 cleaves the 5′ FLAP, the hybridized RTL probes are ligated using methods disclosed herein (e.g., enzymatically using e.g., T4 DNA ligase or chemically). In some instances, the ligation step comprises ligating the first RTL probe and the 3′ end of a gap RTL probe using enzymatic or chemical ligation. In some instances, the ligation step comprises ligating the second RTL probe and the 5′ end of a gap RTL probe using enzymatic or chemical ligation. In some instances, the enzymatic ligation utilizes a ligase. In some instances, the ligase is one or more of a T4 DNA ligase (Rnl2), a SplintR ligase, a single stranded DNA ligase, or a T4 DNA ligase. In some instances, the ligase is a T4 DNA ligase 2 (Rnl2) ligase. In some instances, the ligase is a pre-activated T4 DNA ligase. In some embodiments, after releasing the 5′ FLAP from the second RTL probe, the biological sample is permeabilized. In some embodiments, permeabilization occurs using a protease (e.g., an endopeptidase disclosed herein).

### SNP Detection in a Biological Sample Using a 5′ FLAP

This disclosure also features a method of detecting a genetic variant in gDNA analyte in a biological sample where the method includes determining all or a part of the sequence of the 5′ FLAP, and using the sequence of the 5′ FLAP to detect the genetic variant in the gDNA analyte without using a substrate including capture probes to capture the 5′ FLAP.

In another aspect, this disclosure features a method of detecting a genetic variant in a gDNA analyte in a biological sample including: (a) contacting the gDNA analyte with a first RTL probe and a second RTL probe, wherein the first RTL probe and the second RTL probe each include a sequence that is substantially complementary to adjacent sequences of the gDNA analyte; wherein the first RTL probe and the second RTL probe are capable of forming an invasive cleavage structure in the presence of the genetic variant; and wherein the second RTL probe further includes a 5′ FLAP; (b) hybridizing the first RTL probe and the second RTL probe to the gDNA analyte; (c) cleaving the second RTL probe when the genetic variant is present, thereby releasing the 5′ FLAP; and (d) determining all or a part of the sequence of the 5′ FLAP, and using the sequence of the 5′ FLAP to detect the genetic variant in the gDNA analyte. In some embodiments, the method also includes denaturing the gDNA under conditions wherein the first RTL probe and the second RTL probe can hybridize to the gDNA analyte. In some embodiments, the method includes a denaturing step prior to contacting the gDNA analyte with a first RTL probe and a second RTL probe. In some embodiments, the method further includes contacting the biological sample with a permeabilization reagent. In some embodiments, the biological sample is contacted with the permeabilization reagent determining all or a part of the sequence of the 5′ FLAP, and using the sequence of the 5′ FLAP to detect the genetic variant in the gDNA analyte. In some embodiments, the method also includes a determining step including amplifying all or part of the 5′ FLAP. In some embodiments, the amplifying includes isothermal amplification. In some embodiments, the determining step includes sequencing.

In some embodiments, the cleaving step includes providing an endonuclease. In some embodiments, the endonuclease cleaves the invasive cleavage structure. In some embodiments, the endonuclease cleaves a portion of the first RTL probe. In some embodiments, the endonuclease cleaves the at least one nucleotide that is complementary to a wild-type sequence of the gDNA analyte of the first RTL probe. In some embodiments, the endonuclease cleaves a portion of the second RTL probe. In some embodiments, the endonuclease cleaves the 5′ FLAP of the second RTL probe. In some embodiments, the endonuclease is FLAP endonuclease 1 (FEN1).

In some embodiments, the 5′ FLAP further includes a first barcode sequence. In some embodiments, the first barcode sequence includes a sequence that identifies the first RTL probe, the second RTL probe, and/or the gDNA analyte. When the 5′ FLAP is cleaved, the 5′ FLAP can be used to identify a hybridization event where both the first RTL probe and the second RTL probe hybridize to a gDNA analyte.

In some embodiments, the 5′ FLAP also includes a functional sequence. The functional sequence can be a primer sequence. The primer sequence can be used to amplify the 5′ FLAP before cleavage, contemporaneously with cleavage, or after cleavage of the 5′ FLAP from the second RTL probe. The functional sequence can be a sequence for use in sequencing the FLAP.

In some embodiments, when the method comprises cleaving the second RTL probe when the genetic variant is present, the 5′ FLAP includes a nucleotide that is complementary to the genetic variant. In some embodiments, following cleavage of the 5′ FLAP, the portion of the second RTL probe that is not released includes a nucleotide that is complementary to the genetic variant.

In some embodiments, the second RTL probe includes a second barcode. For example, the second barcode is on the portion of the second RTL probe that is not released following cleavage. In some embodiments, the second barcode can be used to identify the second RTL probe.

This disclosure features methods of detecting a genetic variant in a gDNA analyte at a spatial location in a biological sample using a substrate to capture the released 5′ FLAP. In such cases, the method includes contacting the biological sample with a substrate including a plurality of capture probes, where, a capture probe of the plurality includes a spatial barcode and a capture domain, hybridizing the 5′ FLAP to the capture domain, and determining the sequence of the 5′ FLAP and using the sequence of the 5′ FLAP to detect a genetic variant in a gDNA analyte at a spatial location in the biological sample.

A non-limiting example of a method of detecting a genetic variant in a gDNA analyte at a spatial location in a biological sample includes: (a) contacting the biological sample with a first RTL probe and a second RTL probe, wherein the first RTL probe and the second RTL probe each include a sequence that is substantially complementary to adjacent sequences of the gDNA analyte; wherein the first RTL probe and the second RTL probe are capable of forming an invasive cleavage structure when the genetic variant is present; and wherein the second RTL probe further includes a 5′ FLAP; (b) hybridizing the first RTL probe and the second RTL probe to the gDNA analyte; (c) cleaving the second RTL probe when the genetic variant is present, thereby releasing the 5′ FLAP; (d) contacting the biological sample with a substrate including a plurality of capture probes, wherein a capture probe of the plurality includes a spatial barcode and a capture domain; (e) hybridizing the 5′ FLAP to the capture domain; and (f) determining (i) all or a part of the sequence of the 5′ FLAP, or a complement thereof, and (ii) all or a part of the sequence of the spatial barcode, or a complement thereof, and using the determined sequence of (i) and (ii) to identify the spatial location of the gDNA analyte in the biological sample. In some embodiments, the method also includes denaturing the gDNA under conditions wherein the first RTL probe and the second RTL probe can hybridize to the gDNA analyte. In some embodiments, the method includes a denaturing step prior to contacting the biological sample with a first RTL probe and a second RTL probe. In some embodiments, the method further includes contacting the biological sample with a permeabilization reagent. In some embodiments, the biological sample is contacted with the permeabilization reagent before determining all or a part of the sequence of the 5′ FLAP, and using the sequence of the 5′ FLAP to detect the genetic variant in the gDNA analyte.

In some embodiments, the cleaving step includes contacting with an endonuclease. In some embodiments, the endonuclease cleaves the invasive cleavage structure. In some embodiments, the endonuclease cleaves a portion of the first RTL probe. In some embodiments, the endonuclease cleaves the at least one nucleotide that is complementary to a wild-type sequence of the gDNA analyte of the first RTL probe. In some embodiments, the endonuclease cleaves a portion of second RTL probe. In some embodiments, the endonuclease cleaves the 5′ FLAP of the second RTL probe. In some embodiments, the endonuclease is FLAP endonuclease 1 (FEN1).

In some embodiments, the method also includes providing a capture probe binding domain blocking moiety that interacts with the capture probe binding domain. In some embodiments, the method further includes releasing the capture probe binding domain blocking moiety from the capture probe binding domain prior to contacting the biological sample with the substrate. In some embodiments, the capture probe binding domain blocking moiety includes a poly-uridine sequence, a poly-thymidine sequence, or both. In some embodiments, releasing the capture probe binding domain blocking moiety from the poly(A) sequence includes denaturing the 5′ FLAP.

In some embodiments where the method includes detecting a genetic variant in a gDNA analyte at a spatial location in a biological sample, the determining step includes amplifying all or part of the 5′ FLAP specifically bound to the capture domain. For example, the amplification can be isothermal. In some embodiments, the determining step includes sequencing the 5′ FLAP.

In some embodiments, the method of detecting a genetic variant in a gDNA analyte at a spatial location in a biological sample includes a second RTL probe having a 5′ FLAP that further includes a first barcode sequence. In some embodiments, the first barcode sequence includes a sequence that identifies the first RTL probe, the second RTL probe, and/or the gDNA analyte. In some embodiments, the 5′FLAP further includes a functional sequence. For example, the functional sequence is a primer sequence. In some embodiments, the 5′ FLAP further includes a capture probe binding domain. The capture probe binding domain can include a homopolymeric sequence. For example, without limitation, the capture probe binding domain can be a poly(A) sequence.

In some embodiments, following cleavage of the 5′ FLAP, the 5′ FLAP includes a nucleotide that is complementary to the genetic variant. In some embodiments, following cleavage of the 5′ FLAP, the portion of the second RTL probe that is not released by the cleavage includes a nucleotide that is complementary to the genetic variant.

In some embodiments, the method of detecting a genetic variant in an gDNA analyte at a spatial location in a biological sample includes a second RTL probe that includes from 5′ to 3′: a 5′ FLAP and a sequence that is substantially complementary to a sequence that is adjacent to a sequence that is substantially complementary to a first RTL probe.

In some embodiments, the second RTL probe includes a 5′ FLAP including from 5′ to 3′: a functional sequence, a barcode, and a capture probe binding domain sequence. In some embodiments, the 5′ FLAP includes from 5′ to 3′: a functional sequence, a barcode, a capture probe binding domain sequence, and an additional nucleotide. In some embodiments, the additional nucleotide includes a nucleotide that is complementary to the genetic variant. In some embodiments, the additional nucleotide includes a nucleotide that is complementary to the wild type sequence of the genetic variant. In some embodiments, the second RTL probe further includes a nucleotide that is complementary to the genetic variant.

In some embodiments, the second RTL probe further includes a second barcode. In some embodiments, the second barcode sequence includes a sequence that identifies the first RTL probe, the second RTL probe, and/or the gDNA analyte. In some embodiments, the second barcode sequence includes a sequence that identifies the second RTL probe. A second RTL probe can include from 5′ to 3′: a 5′ FLAP, a sequence that is substantially complementary to a sequence that is adjacent to a sequence that is substantially complementary to a first RTL probe, and a second barcode. In some embodiments, the second RTL probe further includes a second capture probe binding domain. For example, a second RTL probe can include from 5′ to 3′: a 5′ FLAP, a sequence that is substantially complementary to a sequence that is adjacent to a sequence that is substantially complementary to a first RTL probe, a second barcode, and a second capture probe binding domain.

### (c) Hybridization of 5′ FLAP to Capture Domain

In some embodiments, the 5′ FLAP includes a capture probe binding domain, which can hybridize to a capture probe (e.g., a capture probe immobilized, directly or indirectly, on a substrate). In some embodiments, methods provided herein include contacting a biological sample with a substrate, where the capture probe is affixed to the substrate (e.g., immobilized to the substrate, directly or indirectly). In some embodiments, the capture probe includes a spatial barcode and a capture domain. In some embodiments, the capture probe binding domain of the 5′ FLAP specifically binds to the capture domain. After hybridization of the 5′ FLAP to the capture probe, the 5′ FLAP is extended at the 3′ end to make a copy of the additional components (e.g., the spatial barcode) of the capture probe.

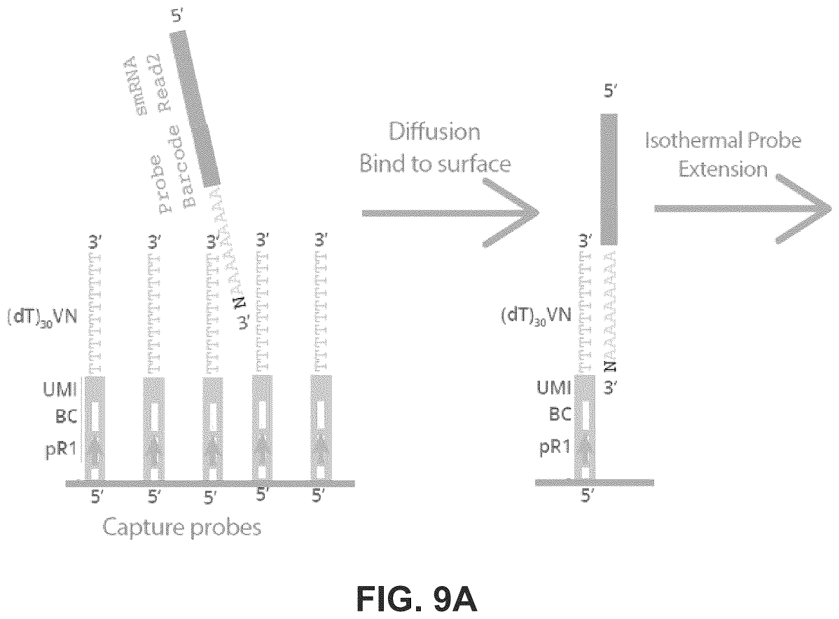

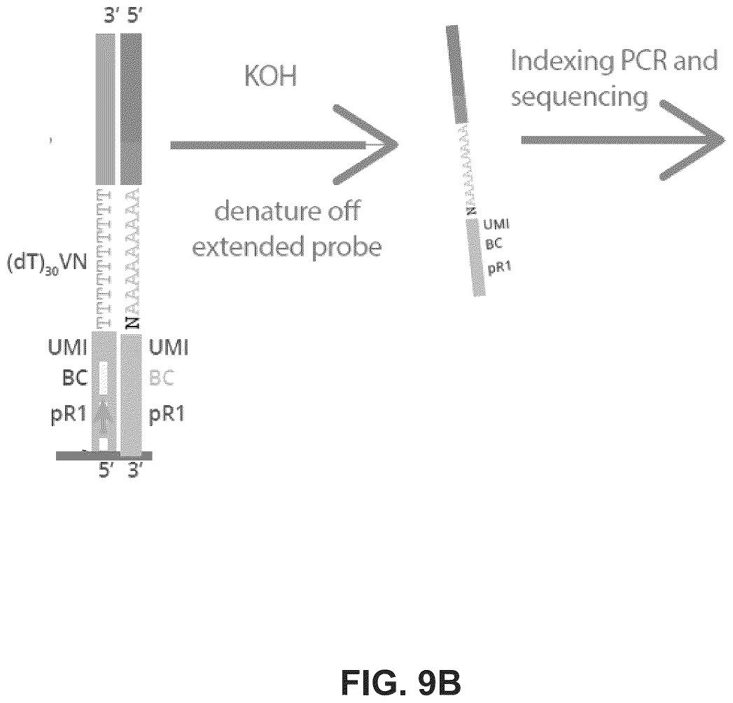

In some embodiments where the 5′ FLAP hybridizes to a capture probe on a substrate, the 5′ FLAP is extended to generate an extended 5′ FLAP. As shown in FIGS. 9A-9B, the 5′ FLAP is extended at the 3′ end to make a copy of the additional components (e.g., the spatial barcode) of the capture probe. In such cases, the 5′ FLAP can include, for example, a sequence complementary to a UMI, spatial barcode, Read 1 sequence (pR1), and a hybridization sequence. In some embodiments, extending the 5′ FLAP includes a polymerase enzyme (e.g., a DNA polymerase or DNA polymerase). In some embodiments, the extension reaction can include an isothermal reaction. For example, the extension reaction can use a Phi29 DNA polymerase to extend the 5′ FLAP. The Phi29 DNA polymerase includes 3′ to 5′ exonuclease activity that can be used to remove one or more additional nucleotides from the 3′ end of a 5′ FLAP. In another example, the extension reaction can use a Bst DNA polymerase to extend the 5′ FLAP.

In some embodiments where the 5′ FLAP includes an additional nucleotide at the 3′ end, extending the 5′ FLAP includes using a polymerase having a 3′ to 5′ exonuclease activity to remove the additional nucleotide sequence from the 3′ end and then extend the 5′ FLAP. The resulting extended 5′ FLAP can be denatured from the capture probe template and transferred (e.g., to a clean tube) for amplification, and/or library construction as described herein. The spatially-barcoded, full-length extended 5′ FLAP can be amplified via PCR prior to library construction. P5, P7, i7, and i5 can be incorporated into the library as for downstream sequencing, and additional library sequencing regions, such as TruSeq Read 2, can be added via End Repair, A-tailing, Adaptor Ligation, and PCR. The cDNA fragments can then be sequenced using paired-end sequencing using TruSeq Read 1 and TruSeq Read 2 as sequencing primer sites. In some instances, the cDNA library is sequenced using any method described herein, such that different sequencing domains specific to other sequencing methods and techniques can be incorporated into a capture probe or introduced during library preparation. In some instances, the sequence of the RTL product is determined via sequencing. In some instances, the spatial barcode is sequenced, providing the location of the gDNA analyte.

### (d) RTL Probe Sets Comprising a 5′ FLAP on One Probe

Provided herein are templated ligation probes, or RTL probes, that hybridize to adjacent sequences of a target gDNA analyte. Compared to situations of templated ligation where the probes are ligated at each one's terminal end, herein, one of the probes includes a sequence that is not complementary to the target gDNA analyte, and thus overhangs on the side adjacent to the other RTL probes upon RTL probe hybridization.

In some instances, a set of RTL probes comprises a first RTL probe and a second RTL probe. In some instances, the first RTL probe comprises a primer sequence and a sequence complementary to a target analyte (e.g., gDNA). In some instances, the second RTL probe includes a 5′ FLAP, a sequence complementary to a target analyte (e.g., gDNA), and a poly(A) sequence. Detailed descriptions of RTL probes has been disclosed in WO 2021/133849, the entirety of which is incorporated herein by reference.

In some embodiments, one of the RTL probes (e.g., the second RTL probe) includes a sequence of non-complementary nucleotides (e.g. a 5′ FLAP). In some embodiments, non-complementary nucleotides include ten nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 51 nucleotides, 52 nucleotides, 53 nucleotides, 54 nucleotides, 55 nucleotides, 56 nucleotides, 57 nucleotides, 58 nucleotides, 59 nucleotides, or at least 60 nucleotides.

In some instances, the 5′ FLAP includes a functional sequence. The functional sequence can be a primer sequence. Other non-limiting examples of functional sequences includes Read 1 or Read 2 sequences (e.g., sequences that can be added for sequencing library preparation), index sequences such as an i5 sequence, and/or an i7 sequence (e.g., sequences complementary to P5 and P7), or any other sequence that facilitates detection of the 5′ FLAP. In some embodiments, the functional sequence can be a primer sequence where a primer hybridizes to the primer sequence and is used to amplify the 5′ FLAP. The 5′ FLAP can be amplified using any form of amplification, for example, linear amplification, PCR, of isothermal amplification, and the like.

In some instances, the 5′ FLAP includes a barcode sequence. The barcode sequence is a nucleic acid sequence that functions as a label or identifier of the 5′ FLAP. The barcode sequence can also function as a label of the first RTL probe, the second RTL probe, and/or the gDNA analyte to which the first RTL probe and the second RTL probe hybridize.

In some instances, the 5′ FLAP includes a capture probe binding domain sequence (e.g., any of the exemplary capture probe binding domain sequences described herein). The capture probe binding domain enables the 5′ FLAP to hybridize to a capture domain on a capture probe. In some cases, the capture probe is located on the surface of a substrate and includes a spatial barcode and a capture domain. In some embodiments, the capture probe binding domain includes a poly-uridine sequence, a poly-thymidine sequence, or both. In some embodiments, the capture probe binding domain includes a poly(A) sequence. In some embodiments, the capture probe binding domain includes a random sequence (e.g., a random hexamer or octamer). In some embodiments, the capture probe binding domain is complementary to a capture domain in a capture probe that detects a particular target(s) of interest. In some instances, the 5′ FLAP does not include a capture probe binding domain sequence. In this instance, identification of the 5′ FLAP is performed via downstream analysis (e.g., sequencing the 5′ FLAP or a complement thereof without hybridization to a probe on an array).

In some embodiments, the 5′ FLAP includes an additional nucleotide on its 3′ end. For example, the 5′ FLAP includes from 5′ to 3′: a functional sequence, a barcode, a capture probe binding domain sequence, and an additional nucleotide. The additional nucleotide can include a nucleotide that is complementary to the target sequence (e.g., the genetic variant). Alternatively, the additional nucleotide can include a nucleotide that is complementary to the target sequence (e.g., wild type sequence of the genetic variant). In some embodiments, the 5′ FLAP includes an additional two, there, four or five or more nucleotides on its 3′ end.

In some embodiments, the subset of analytes includes an individual target DNA. In some instances, the presence of the 5′ FLAP that is created as a result of the methods described herein indicates that the individual target DNA is present. In some instances, the absence of the 5′ FLAP that is created as a result of the RTL methods described herein indicates that the individual target DNA is present. In some instances, an absence of the 5′ FLAP is because one of the RTL probes did not hybridize to the gDNA analyte. In some instances, an absence of the 5′ FLAP is because both (e.g., two) of the RTL probes did not hybridize to the gDNA analyte.

In some embodiments, the subset of analytes detected using methods disclosed herein includes two or more (e.g., 2, 3, 4, 5, 6, 7, 8, 9, 10, or more) targeted DNAs. In some instances, the detection of analytes includes detection of one or more mutations in a gDNA analyte. In some embodiments, the subset of analytes includes detection of gDNAs having one or more single nucleotide polymorphisms (SNPs) in a biological sample.

In some embodiments, the methods that allow for targeted DNA capture as provided herein include a first RTL probe and a second RTL probe. The first and second RTL probes each include sequences that are substantially complementary to the sequence of an analyte of interest. By substantially complementary, it is meant that the first and/or second RTL probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a sequence in an DNA molecule. In some instances, the first RTL probe and the second RTL probe hybridize to adjacent sequences on a gDNA analyte. It is appreciated that the terms first RTL probe and the second RTL probe generally can be used interchangeably.

In some embodiments, the first and/or second probe as disclosed herein include a combination of ribonucleic acids and deoxyribonucleic acids. In some instances of the first and/or second RTL probes, the sequence that is substantially complementary to the sequence of the analyte of interest includes ribonucleic acids (e.g., does not include deoxyribonucleic acids). In some instances of the first and/or second RTL probes, the sequence that is substantially complementary to the sequence of the analyte of interest includes deoxyribonucleic acids (e.g., does not include ribonucleic acids).

In some embodiments, the first and/or second RTL probe as disclosed herein includes one of at least two ribonucleic acid bases at the 3′ end; a functional sequence; a phosphorylated nucleotide at the 5′ end; and/or a capture probe binding domain. In some embodiments, the first and/or second RTL probe as disclosed herein includes one of at least two deoxyribonucleic acid bases at the 3′ end; a functional sequence; a phosphorylated nucleotide at the 5′ end; and/or a capture probe binding domain. In some embodiments, the functional sequence is a primer sequence. The capture probe binding domain is a sequence that is complementary to a particular capture domain present in a capture probe. In some embodiments, the capture probe binding domain includes a poly(A) sequence. In some embodiments, the capture probe binding domain includes a poly-uridine sequence, a poly-thymidine sequence, or both. In some embodiments, the capture probe binding domain includes a random sequence (e.g., a random hexamer or octamer). In some embodiments, the capture probe binding domain is complementary to a capture domain in a capture probe that detects a particular target(s) of interest.

In some embodiments, a capture probe binding domain blocking moiety that interacts with the capture probe binding domain is provided. In some instances, the capture probe binding domain blocking moiety includes a nucleic acid sequence. In some instances, the capture probe binding domain blocking moiety is a DNA oligonucleotide. In some instances, the capture probe binding domain blocking moiety is an RNA oligonucleotide. In some embodiments, a capture probe binding domain blocking moiety includes a sequence that is complementary or substantially complementary to a capture probe binding domain. In some embodiments, a capture probe binding domain blocking moiety prevents the capture probe binding domain from binding the capture probe when present. In some embodiments, a capture probe binding domain blocking moiety is removed prior to binding the capture probe binding domain (e.g., present in a 5′ FLAP) to a capture probe. In some embodiments, a capture probe binding domain blocking moiety comprises a poly-uridine sequence, a poly-thymidine sequence, or both.

### (e) Biological Samples

Methods disclosed herein can be performed on any type of biological sample, or sample. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample. In certain embodiments, methods provided herein enable sensitive measurement of specific genes of interest that otherwise might be missed with a whole transcriptomic approach.

In some embodiments, a biological sample (e.g. tissue section) can be fixed with methanol, stained with hematoxylin and eosin, and imaged. In some embodiments, fixing, staining, and imaging occurs before one or more oligonucleotide probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

### (f) Compositions and Kits

In some instances, disclosed herein are compositions and systems that are used to carry out the methods described herein. In some instances, the kit includes a first RTL probe and a second RTL probe. In some instances, the first RTL probe and the second RTL probe in the kit are substantially complementary to adjacent sequences of an analyte. In some instances, the second RTL probe includes a 5′ FLAP that is not complementary to the analyte.

In some instances, the kit includes multiple sets of RTL probes, allowing for detection of multiple analytes using one kit. For example, in some instances, the kit includes at least 2 sets, at least 3 sets, at least 4 sets, at least 5 sets, at least 6 sets, at least 7 sets, at least 8 sets, at least 9 sets, or at least 10 sets, or more sets of RTL probes. It is appreciated that multiple sets of probes can detect nucleic acids or variants that are associated with particular physiologies or pathophysiologies (e.g., cancer).

In some instances, the kit includes one or more enzymes, including an endonuclease. In some instances, the endonuclease is FEN1. In some instances, the endonuclease cleaves the 5′ FLAP thereby releasing the 5′ FLAP from the second RTL probe.

In some instances, the kit further includes a substrate comprising a plurality of capture probes, wherein a capture probe of the plurality comprises a spatial barcode and a capture domain, wherein the 5′ FLAP is capable of hybridizing to the capture domain. It is appreciated that the kit can include any of the elements of the substrate, array, or capture probes as described herein.

In some instances, the kit further includes instructions for carrying out the methods of detecting a 5′ FLAP as described herein.

In some instances, compositions and systems are further provided herein. In some instances, a composition or system provided herein includes a first RTL probe and a second RTL probe hybridized to an analyte, wherein the first RTL probe and the second RTL probe are substantially complementary to adjacent sequences of the analyte, and wherein the second RTL probe comprises a 5′ FLAP. In some instances, a composition or system provided herein includes a first RTL probe and a second RTL probe hybridized to an analyte, wherein the first RTL probe and the second RTL probe each comprise a sequence that is substantially complementary to adjacent sequences of the gDNA analyte; wherein the first RTL probe and the second RTL probe are capable of forming an invasive cleavage structure in the presence of the genetic variant; and wherein the second RTL probe further comprises a 5′ FLAP. In some instances, the 5′ FLAP comprises a sequence that is capable of hybridizing to a capture domain of a capture probe. In some instances, the capture probe further comprises a spatial barcode. In some instances, the capture probe is part of a plurality of capture probes affixed to any one of the substrates or arrays as described throughout this disclosure.

### EXAMPLES

### Example 1—Detecting an gDNA Analyte in a Biological Sample Using a 5′ FLAP

This example provides an exemplary method of determining the presence or absence of an analyte in a biological sample. In a non-limiting example, a first RTL probe and a second RTL probe are substantially complementary to adjacent sequences of the gDNA analyte, and wherein the second RTL probe includes a 5′ FLAP. In the presence of the gDNA analyte in the biological sample, the first and second probes hybridize to the gDNA analyte, the second RTL probe is cleaved thereby releasing the 5′ FLAP. The sequence of the 5′ FLAP is determined and the determined sequence of the 5′ FLAP is used to detect the gDNA analyte.

As shown in FIG. 5, a gDNA analyte is contacted with a first RTL probe (LHS) and a second RTL probe (RHS). The first RTL probe includes a sequence that is substantially complementary to a sequence that is 3′ of a target sequence. The second RTL probe includes a 5′ FLAP sequence (e.g., a smRNA R2 handle, and a barcode) and a sequence that is substantially complementary to a sequence that is 5′ of the target sequence. Prior to hybridization, the gDNA analyte is denatured under conditions to enable hybridization the RTL probes to the gDNA analyte. The first RTL probe and the second RTL probe can then hybridize to the gDNA analyte. Excess first and second probes are washed off. FEN1 endonuclease cleaves the 5′ FLAP thereby removing the 5′ FLAP from the second RTL probe. The cleavage occurs when the first RTL probe hybridizes to a sequence that is adjacent to a sequence to which the second RTL probe is hybridized. Following FEN1-mediated cleavage, all or part of the sequence of the 5′ FLAP is determined and used to detect a gDNA analyte in a biological sample. Detection can occur via florescence (e.g., FRET pair and release of the FLAP resulting in fluorescence detection).

### Example 2—Detecting a Genetic Variant in a gDNA Analyte in a Biological Sample Using a 5′ FLAP

This example provides an exemplary method for determining the presence or absence of a genetic variant in a gDNA analyte in a biological sample. In a non-limiting example, a first RTL probe having a non-complementary nucleotide overlapping the target sequence (e.g., genetic variant) and a second RTL probe having a sequence complementary to the target sequence (e.g., genetic variant) are used to determine the presence or absence of a genetic variant in a biological sample. Others have demonstrated SNP detection using invader assays. Non-limiting aspects of SNP detection with invader assays are described in U.S. Pat. Nos. 7,011,944, 6,913,881, 6,875,572 and 6,872,816, each of which is incorporated by reference in its entirety and each of which can be used herein in any combination.

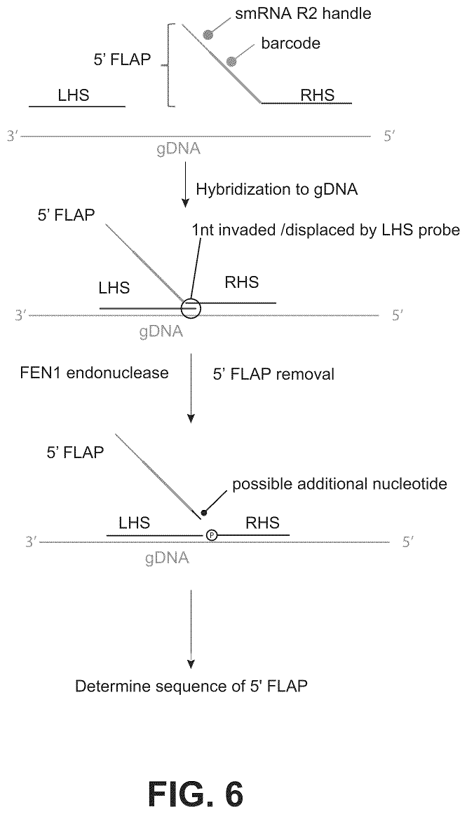

As shown in FIG. 6, a gDNA analyte that includes a single nucleotide polymorphism (SNP) (i.e., genetic variant) at a target sequence is contacted with a first RTL probe and a second RTL probe. The first RTL probe (LHS) includes a sequence that is substantially complementary to a sequence that is 3′ of a target sequence (e.g., genetic variant) and a non-complementary nucleotide that is overlapping the target sequence (e.g., genetic variant). The second RTL probe (RHS) includes a 5′ FLAP sequence (e.g., a smRNA R2 handle and a barcode), a nucleotide that is complementary to the target sequence (e.g., genetic variant), and a sequence that is substantially complementary to a sequence 5′ of the target sequence (e.g., genetic variant). Prior to hybridization, the gDNA analyte is denatured under conditions to enable hybridization of the RTL probes to the gDNA analyte. The first RTL probe and the second RTL probe can then hybridize to the gDNA analyte. Excess first and second probes are washed off. FEN1 endonuclease cleaves the 5′ FLAP thereby removing the non-complementary nucleotides from the second RTL probe. The cleavage occurs when the first RTL probe includes a non-complementary sequence that overlaps with the target sequence (e.g., genetic variant and/or SNP) and the second RTL probe includes a sequence that overlaps with the target sequence and is complementary. In the presence of the genetic variant (e.g., SNP), the first RTL probe and the second RTL probe are then capable of forming an invasive cleavage structure, which is cleaved by FEN1. When the sequence of the second RTL probe is not complementary to the target sequence (e.g., genetic variant) or the first RTL probe includes a complementary sequence that overlaps the target sequence (e.g., genetic variant), no invasive cleavage structure is formed and FEN1-mediated cleave will not occur. Following FEN1-mediated cleavage, all or part of the sequence of the 5′ FLAP is sequenced used to determine the presence or absence of a single nucleotide polymorphism (SNP) at the target sequence. Detection can occur via florescence (e.g., FRET pair and release of the FLAP resulting in fluorescence detection).

### Example 3—Detecting a gDNA Analyte at a Location in a Biological Sample Using a 5′ FLAP and a Substrate

This example provides an exemplary method for determining the location of an analyte in a biological sample using a first RTL probe and a second RTL probe.

In a non-limiting example, a first RTL probe and a second RTL probe are substantially complementary to adjacent sequences of the gDNA analyte, and wherein the second RTL probe includes a 5′ FLAP. In the presence of the gDNA analyte in the biological sample, the first and second probes hybridize to the gDNA analyte, the second RTL probe is cleaved thereby releasing the 5′ FLAP. The sequence of the 5′ FLAP is used to detect the gDNA analyte.

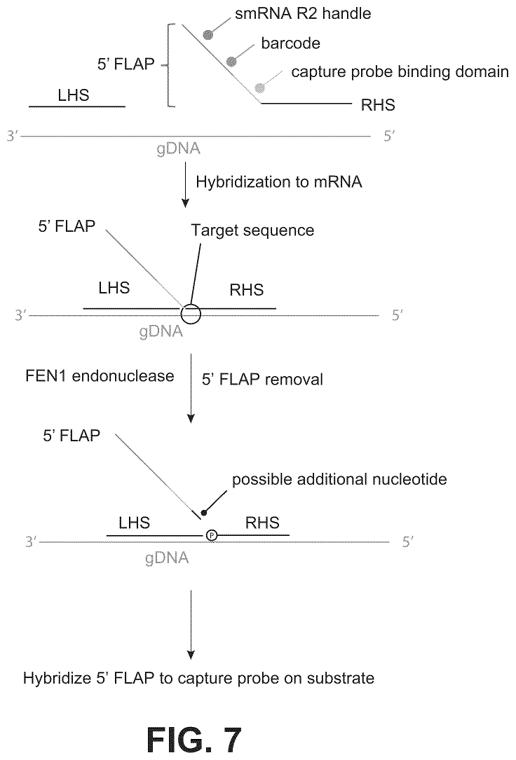

As shown in FIG. 7, a gDNA analyte is contacted with a first RTL probe and a second oligonucleotide. The first RTL probe (LHS) includes a sequence that is substantially complementary to a sequence that is 3′ of a target sequence. The second RTL probe (RHS) includes a 5′ FLAP sequence (e.g., smRNA R2 handle, barcode, and a capture probe binding domain (e.g., a poly(A) sequence)) and a sequence that is complementary to the target sequence and is substantially complementary to a sequence that is 5′ of the target sequence. Prior to hybridization, the gDNA analyte is denatured under conditions to enable hybridization of the RTL probes to the gDNA analyte. The first RTL probe and the second RTL probe can hybridize to the gDNA analyte. Excess first and second probes are washed off. FEN1 endonuclease cleaves the 5′ FLAP thereby removing the non-complementary nucleotides from the second RTL probe. The cleavage occurs when the first RTL probe hybridizes to a sequence that is adjacent to the second RTL probe.

As shown in FIGS. 9A-9B, the biological sample is contacted with a substrate that includes capture probes affixed to the substrate, where the capture probes include a spatial barcode and the capture domain. Following cleavage, the capture probe binding domain of the 5′ FLAP specifically binds to the capture domain of the capture probe, thereby capturing the 5′ FLAP on the substrate. After hybridization of the 5′ FLAP to the capture probe, the 5′ FLAP is extended at the 3′ end to make a copy of the additional components (e.g., the spatial barcode) of the capture probe. Finally, all or part of the sequence of the 5′ FLAP specifically bound to the capture domain along with the all or part of the sequence of the spatial barcode of the capture probe is sequenced and used to determine the presence or absence of a single nucleotide polymorphism (SNP) at the target sequence.

### Example 4—Detecting a Genetic Variant in a gDNA Analyte at a Location in a Biological Sample Using a 5′ FLAP and a Substrate

This example provides an exemplary method for determining the presence or absence of a genetic variant in a gDNA analyte in a biological sample. In a non-limiting example, a first RTL probe having a non-complementary nucleotide overlapping the target sequence (e.g., genetic variant) and a second RTL probe having a sequence complementary to the target sequence (e.g., genetic variant) are used to determine the location of a genetic variant in a biological sample.

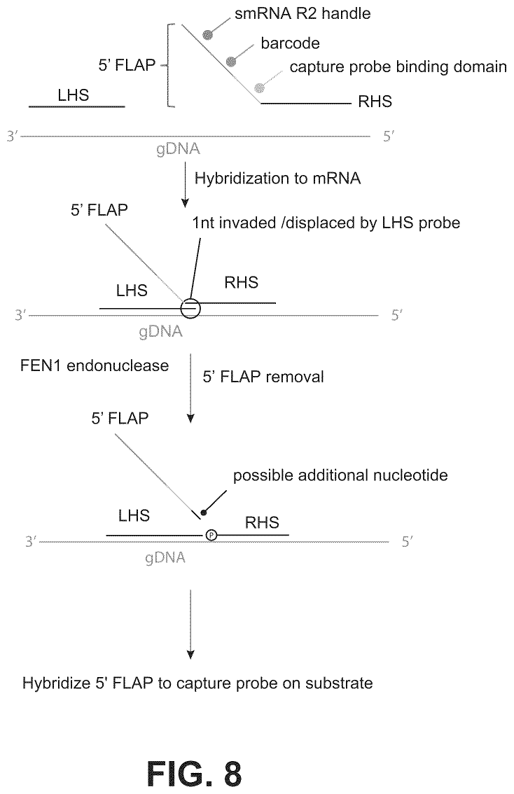

As shown in FIG. 8, a gDNA analyte that includes a single nucleotide polymorphism (SNP) (i.e., genetic variant) at a target sequence is contacted with a first RTL probe and a second RTL probe. The first RTL probe (LHS) includes a sequence that is substantially complementary to a sequence that is 3′ of a target sequence (e.g., genetic variant) and a non-complementary nucleotide that is overlapping the target sequence (e.g., genetic variant). The second RTL probe (RHS) includes a 5′ FLAP sequence (e.g., smRNA R2 handle, barcode, and a capture probe binding domain (e.g., a poly(A) sequence)), a nucleotide that is complementary to the target sequence (e.g., genetic variant), and a sequence that is substantially complementary to a sequence 5′ of the target sequence (e.g., genetic variant). Prior to hybridization, the gDNA analyte is denatured under conditions to enable hybridization of the RTL probes to the gDNA analyte. The first RTL probe and the second RTL probe can hybridize to the gDNA analyte. Excess first and second probes are washed off. FEN1 endonuclease cleaves the 5′ FLAP thereby removing the non-complementary nucleotides from the second RTL probe. The cleavage occurs when the first RTL probe includes a non-complementary sequence that overlaps with the target sequence (e.g., genetic variant and/or SNP) and the second RTL probe includes a sequence that overlaps with the target sequence and is complementary. In the presence of the genetic variant (e.g., SNP), the first RTL probe and the second RTL probe are then capable of forming an invasive cleavage structure, which is cleaved by FEN1. When the sequence of the second RTL probe is not complementary to the target sequence (e.g., genetic variant) or the first RTL probe includes a complementary sequence that overlaps the target sequence (e.g., genetic variant), no invasive cleavage structure is formed and FEN1-mediated cleave will not occur.

As shown in FIGS. 9A-9B, the biological sample is contacted with a substrate that includes capture probes affixed to the substrate, where the capture probes include a spatial barcode and the capture domain. Following cleavage, the capture probe binding domain of the 5′ FLAP specifically binds to the capture domain of the capture probe, thereby capturing the 5′ FLAP on the substrate. After hybridization of the 5′ FLAP to the capture probe, the 5′ FLAP is extended at the 3′ end to make a copy of the additional components (e.g., the spatial barcode) of the capture probe. Finally, all or part of the sequence of the 5′ FLAP specifically bound to the capture domain along with the all or part of the sequence of the spatial barcode of the capture probe is sequenced and used to determine the presence or absence of a single nucleotide polymorphism (SNP) at the target sequence.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. In case of conflict, the present specification, including definitions, will control. In addition, section headings, the materials, methods, and examples are illustrative only and not intended to be limiting.

What is claimed is:

1. A method for determining the abundance and/or location of a genomic DNA (gDNA) analyte in a biological sample, the method comprising:
(a) contacting the biological sample with a substrate comprising a plurality of capture probes, wherein a capture probe of the plurality comprises a spatial barcode and a capture domain;
(b) contacting the biological sample with a first RTL probe and a second RTL probe, wherein the first RTL probe and the second RTL probe are substantially complementary to adjacent sequences of the gDNA analyte, and wherein the second RTL probe comprises a 5′ FLAP;
(c) hybridizing the first RTL probe and the second RTL probe to the gDNA analyte;
(d) cleaving the second RTL probe, thereby releasing the 5′ FLAP;
(e) hybridizing the 5′ FLAP to the capture domain; and
(f) determining (i) all or a part of the sequence of the 5′ FLAP, or a complement thereof, and (ii) all or a part of the sequence of the spatial barcode, or a complement thereof, and using the determined sequence of (i) and (ii) to identify the abundance and/or location of the gDNA analyte in the biological sample.

2. The method of claim 1, wherein the first RTL probe and the second RTL probes are DNA probes.

3. The method of claim 1, wherein the 5′ FLAP comprises a first barcode sequence, wherein the first barcode sequence comprises a sequence that identifies the first RTL probe, the second RTL probe, the gDNA analyte, or any combination thereof.

4. The method of claim 1, wherein the 5′ FLAP comprises (i) a functional sequence, wherein the functional sequence is a primer sequence, and (ii) a capture probe binding domain, wherein the capture probe binding domain comprises a homopolymeric sequence or a poly(A) sequence.

5. The method of claim 1, wherein the cleaving the second RTL probe comprises providing an endonuclease, wherein the endonuclease cleaves a portion of the first RTL probe, a portion of second RTL probe, the 5′ FLAP of the second RTL probe, or any combination thereof.

6. The method of claim 1, wherein the determining step comprises sequencing all or part of the 5′ FLAP.

7. The method of claim 1, wherein the biological sample is a formalin-fixed, paraffin-embedded (FFPE) sample.

8. A method of detecting a genetic variant in a gDNA analyte in a biological sample, the method comprising:
(a) contacting the biological sample with a substrate comprising a plurality of capture probes, wherein a capture probe of the plurality comprises a spatial barcode and a capture domain;
(b) contacting the biological sample with a first RTL probe and a second RTL probe, wherein the first RTL probe and the second RTL probe each comprise a sequence that is substantially complementary to adjacent sequences of the gDNA analyte; wherein the first RTL probe and the second RTL probe are capable of forming an invasive cleavage structure in the presence of the genetic variant; and wherein the second RTL probe further comprises a 5′ FLAP;
(c) hybridizing the first RTL probe and the second RTL probe to the gDNA analyte;
(d) cleaving the second RTL probe when the genetic variant is present, thereby releasing the 5′ FLAP;
(e) hybridizing the 5′ FLAP to the capture domain; and
(f) determining (i) all or a part of the sequence of the 5′ FLAP, or a complement thereof, and (ii) all or a part of the sequence of the spatial barcode, or a complement thereof, and using the determined sequence of (i) and (ii) to detect the genetic variant in the gDNA analyte in the biological sample.

9. The method of claim 8, wherein the genetic variant is a single nucleotide polymorphism (SNP) or a nucleotide point mutation, or wherein the genetic variant comprises at least two, at least three, at least four, at least five, or more genetic variants.

10. The method of claim 8, wherein the first RTL probe comprises a sequence that is substantially complementary to a sequence 3′ to the genetic variant, or at least one nucleotide that is complementary to a wild-type sequence of the genetic variant.

11. The method of claim 8, wherein the second RTL probe comprises a sequence substantially complementary to a sequence 5′ to the genetic variant, and/or a nucleotide that is complementary to the genetic variant.

12. The method of claim 8, wherein the 5′ FLAP further comprises a nucleotide that is complementary to the genetic variant.

13. The method of claim 8, wherein the first RTL probe and the second RTL probe are capable of forming an invasive cleavage structure in the presence of the genetic variant.

14. The method of claim 8, wherein the 5′ FLAP further comprises a capture probe binding domain, wherein the capture probe binding domain comprises a homopolymeric sequence or a poly(A) sequence.

15. The method of claim 12, wherein the 5′ FLAP comprises from 5′ to 3′; a functional sequence, a barcode, and a capture probe binding domain sequence.

16. The method of claim 12, wherein the 5′ FLAP comprises from 5′ to 3′; a functional sequence, a barcode, a capture probe binding domain sequence, and an additional nucleotide, wherein the additional nucleotide comprises a nucleotide that is complementary to the genetic variant or a nucleotide that is complementary to the wild type sequence of the genetic variant.

17. The method of claim 12, wherein the method further comprises providing a capture probe binding domain blocking moiety that interacts with the capture probe binding domain, wherein the method further comprises releasing the capture probe binding domain blocking moiety from the capture probe binding domain prior to contacting the biological sample with the substrate, wherein the capture probe binding domain blocking moiety comprises a poly-uridine sequence, a poly-thymidine sequence, or both, and wherein releasing the capture probe binding domain blocking moiety from the poly(A) sequence comprises denaturing the ligated probe.

18. The method of claim 1, wherein the biological sample is an FFPE sample.

19. A kit comprising:
(a) a first RTL probe and a second RTL probe, wherein the first RTL probe and the second RTL probe are substantially complementary to adjacent sequences of a gDNA analyte, or wherein the first RTL probe and the second RTL probe each comprise a sequence that is substantially complementary to adjacent sequences of the gDNA analyte, and wherein the second RTL probe comprises a 5′ FLAP;
(b) an endonuclease, wherein the endonuclease cleaves the 5′ FLAP thereby releasing the 5′ FLAP from the second RTL probe;
(c) a substrate comprising a plurality of capture probes, wherein a capture probe of the plurality comprises a spatial barcode and a capture domain; and
(d) instructions for performing the method of claim 1.

20. A composition for determining the abundance and/or location of a gDNA analyte in a biological sample, wherein the composition comprises:
a first RTL probe and a second RTL probe hybridized to the gDNA analyte, wherein the first RTL probe and the second RTL probe are substantially complementary to adjacent sequences of the gDNA analyte, or wherein the first RTL probe and the second RTL probe each comprise a sequence that is substantially complementary to adjacent sequences of the gDNA analyte, and
wherein the second RTL probe comprises a 5′ FLAP, wherein the 5′ FLAP comprises a sequence that is capable of hybridizing to a capture domain of a capture probe.
